SEQUENCE LISTING (24 sequences)

SEQ ID NO: 1 - DNA (cDNA), 34 bp; single-stranded, linear
  immediate source clone: OWB260 PRIMER
gagagaaagcttgttgtgcgagaggccaagtggg

SEQ ID NO: 2 - DNA (cDNA), 35 bp; single-stranded, linear
  anti-sense: YES
  immediate source clone: OWB259 primer
gagagaggatcctgcaagatccatgtcgtgctttc

SEQ ID NO: 3 - DNA (cDNA), 29 bp; single-stranded, linear
  anti-sense: NO
  immediate source clone: OWB240 primer
aatgagctctcatggctaagtttgcttcc

SEQ ID NO: 4 - DNA (cDNA), 29 bp; single-stranded, linear
  anti-sense: YES
  immediate source clone: OWB241 primer
aatccatggaatacacacgatttagcacc

SEQ ID NO: 5 - DNA (cDNA), 23 bp; single-stranded, linear
  anti-sense: NO
  immediate source clone: OWB270 PRIMER
ggcgatgaagctcaatccaaacg

SEQ ID NO: 6 - DNA (cDNA), 25 bp; single-stranded, linear
  anti-sense: YES
  immediate source clone: OWB271 primer
ggtcacgaccagcaagatcaagacg

SEQ ID NO: 7 - DNA (cDNA), 35 bp; single-stranded, linear
  immediate source clone: OWB257 primer
gagagaggatccaacttctgtgcttccaccattgc

SEQ ID NO: 8 - DNA (cDNA), 36 bp; single-stranded, linear
  immediate source clone: OWB256 Primer
gagagaaagcttgaagccaagtgggacatggtcagg

SEQ ID NO: 9 - DNA (cDNA), 37 bp; single-stranded, linear
  immediate source clone: OWB255 primer
gagagaggatccagcagcaaagagaacaagagcagcg

SEQ ID NO: 10 - DNA (cDNA), 37 bp; single-stranded, linear
  immediate source clone: OWB273 primer
aagaaagcttatgcatgcatcgccgcatcgatatccc

SEQ ID NO: 11 - DNA (cDNA), 43 bp; single-stranded, linear
  immediate source clone: OWB276 primer
gagagaagcttatttttatgtaaaatacacacgatttagcacc

SEQ ID NO: 12 - DNA (cDNA), 35 bp; single-stranded, linear
  immediate source clone: OWB272 primer
gagagaggatcctgatggaagcaaacttagccatg

SEQ ID NO: 13 - DNA (cDNA), 403 bp; single-stranded, linear
  original source strain: PDF 1.1
aaacaactaagtataaacaatagtcatggctaagtctgctaccatcgttactcttttcttcgctgctcttgttttctttgctgctcttgaagcaccgatggtggtggaagcacagaagttgtgcgagaggccaagtgggacatggtccggagtttgcggaaacagtaacgcgtgcaagaatcagtgcattaaccttgagaaagcacgacatggatcttgcaactatgtcttcccagctcacaaatgtatctgctacttcccatgttaatctaccaagagctcttaatgcttaattataaagtgtgtatttctattaaataagtatgtgtcactctatgagaggtcttatgacatgtaccagatatgttatgttgctttgttttaataaggtaaacttttacgg

SEQ ID NO: 14 - protein, 80 aa
  original source strain: PDF1.1
MAKSATIVTLFFAALVFFAALEAPMVVEAQKLCERPSGTWSGVCGNSNACKNQCINLEKARHGSCNYVFPAHKCICYFPC

SEQ ID NO: 15 - DNA (cDNA), 400 bp; single-stranded, linear
  original source strain: PDF1.2
atacattgaaaacaaaatagtaataatcatcatggctaagtttgcttccatcatcacccttatcttcgctgctcttgttctctttgctgctttcgacgcaccggcaatggtggaagcacagaagttgtgcgagaagccaagtgggacatggtcaggggtttgcggaaacagtaatgcatgcaagaatcagtgcattaaccttgaaggagccaaacatggatcatgcaactatgtcttcccagcacacaagtgtatctgttacgtcccatgttaaatctaccactaatctttggtgctaaatcgtgtgtattttacataaaaataagtcnctgtcactctntgagtaactttatgacatgcatatttctggttttaattgnttattttnccgccggttggt

SEQ ID NO: 16 - protein, 80 aa
  original source strain: PDF 1.2
MAKFASIITLIFAALVLFAAFDAPAMVEAQKLCEKPSGTWSGVCGNSNACKNQCINLEGAKHGSCNYVFPAHKCICYVPC

SEQ ID NO: 17 - protein, 51 aa
  immediate source clone: PDF1.1
QKLCERPSGTWSGVCGNSNACKNQCINLEKARHGSCNYVFPAHKCICYFPC

SEQ ID NO: 18 - protein, 51 aa
  immediate source clone: PDF1.2
QKLCEKPSGTWSGVCGNSNACKNQCINLEGAKHGSCNYVFPAHKCICYVPC

SEQ ID NO: 19 - protein, 51 aa
  immediate source clone: Rs-AFP1
QKLCERPSGTWSGVCGNNNACKNQCINLEKARHGSCNYVFPAHKCICYFPC

SEQ ID NO: 20 - protein, 50 aa
  immediate source clone: Rs-AFP3
KLCERSSGTWSGVCGNNNACKNQCIRLEGAQHGSCNYVFPAHKCICYFPC

SEQ ID NO: 21 - DNA (genomic), 1616 bp; single-stranded, linear
  original source strain: Arabidopsis PDF1.2 gene
  original source individual isolate: DNA sequence Figure 14
atgcatgcatcgccgcatcgatatcccgaggtgcatcgtttctactttcatgtccaaatttggggttaactcacaatatatatcatgttgcctatgtaaatttataatcataaatctaaacccaaattttgatcctcattccaaagcaaaagttctaagccctacaaaaatatgtatttcccaagtttaaaaagaattaatctatacttttacaaatttaaattctgatctcttataatgttcggtttttccttttttatttattaagttagtttaaatttgcagttattttgttgaatgtcgttgtttacgaatttacgaataatacctttatagctaatctacaaaattttgatgactgacaacaccgttaatgtttttttttaaattaccctgagcctctcacttgcggtcagaccatgcatgtcgatagtccattacgtttaaggccacaatcaactatagtttgtttatcaatagccaactaagctaacttttaggttcctgccctctccgttcctccggtaccaatcgtttctttgtcccttcgatagtttgaaaacctaccgacggtgagagcagaatattgatgaatcatccaattttcagtaataggtgtgtcccagggatatataaatggcgaaactgcgcgagaacggttccttgttctgcaaacttggcggaacaatgctgctcttgagatcaaccaaaccatatgtttagtccacaacgatctatatgtctaggggtgatcctctaatcgaaaaatgttgtatttgttcgacgatgacgaaggtcagactatgaactgcacagtctgcgcttgtcctaaccgcgagaatctctgacatcaatatacttgtgtaactatggcttggttaagatattattttcttgagtcttaatccattcagattaaccagccgcccatgtgaacgatgtagcattagctaaaagccgaagcagccgcttaggttactttagatatcgacagagaaatatatgtggtggagaaaccagccatcaacaaacaaaaagcaagatcttatcttttgatattggctacgggaagatgatgtctgtttaatgtgtggggttaccacgttattgtacgatgcacaagtagaagattaacccactaccatttcattataaatagacgttgatctttggcttatttcttcacacaacacatacatctatacattgaaaacaaaatagtaataatcatcatggctaagtttgcttccatcatcacccttatcttcgctgctcttgttctctttgctgctttcggtgagtaataatgttcttattggaaatgtgtttaaatgtaattttagtttatttgataattattcttgactgatgtatgcatatatatgcagacgcaccggcaatggtggaagcacagaagttgtgcgagaagccaagtgggacatggtcaggggtttgcggaaacagtaatgcatgcaagaatcagtgcattaacctcgaaggagccaaacatggatcatgcaactatgtcttcccagcacacaagtgtatctgttacgtcccatgttaaatctaccaataatctttggtgctaaatcgtgtgtattttacataaaaat

SEQ ID NO: 22 - protein, 80 aa; identical to SEQ ID NO: 16
  original source strain: Arabidopsis PDF1.2 amino acid sequence

SEQ ID NO: 23 - protein, 17 aa
MAKFASIRIPGYGQSLM

SEQ ID NO: 24 - DNA (cDNA), 20 bp; single-stranded, linear
tcacgggttggggtttctac

(19) United States
(12) Patent Application Publication
(10) Pub. No.: US 2002/0035738 A1
(43) Pub. Date: Mar. 21, 2002

(54) Plant protection method

(75) Inventors: Bart Pierre Helene Joseph Thomma, Leuven (BE); Franky Raymond Gerard Terras, Anzegem (BE); Iris Anne Marie Armande Penninckx, Heverlee (BE); John Michael Manners, Queensland (AU); Kemal Kazan, Queensland (AU); Willem Frans Broekaert, Dilbeek (BE)

Correspondence Address: ZENECA Ag Products Inc., Intellectual Property Section, 2 Righter Parkway, P.O. Box 15458, Wilmington, DE, 19850-5458, US

(73) Assignee: ZENECA Limited

(21) Appl. No.: 09/732,561

(22) Filed: Dec. 8, 2000

Related U.S. Application Data
(62) Division of application No. 09/202,638, filed on Dec. 18, 1998, now abandoned.
Application No. 09/202,638 is a 371 of international application No. PCT/GB97/01672, filed on Jun. 20, 1997.

(30) Foreign Application Priority Data
Jul. 1, 1996 (GB) 9613753.4

(51) Int. Cl. A01H 5/00

(52) U.S. Cl. 800/279; 800/283

(57) ABSTRACT

A method of protecting a plant against a pathogen is described, the method comprising inducing expression of a plant defensin gene by stimulating the jasmonate and/or ethylenc pathways. Also deseribed is a method of inducing expression of a plant defensin gene, a composition which is capable of inducing expression of a plant defensin gene and a method for screening compounds giving resistance-inducing activity. Preferably, the pathogen is a necrotrophic pathogen.

The present invention relates to a method of protecting a plant against pathogens.

More particularly, the invention relates to a novel signal transduction pathway which leads to expression of proteins which are capable of protecting plants from attack by such pathogens.

In particular, the present invention relates to a method of protecting a plant against necrotrophic pathogens such as fungi and bacteria.

It is well known that plants attacked by microbial pathogens are able to induce the expression of a set of defence-related genes encoding pathogenesis-related proteins (PR proteins). These proteins can be detected not only at the site of pathogen attack but also in non-infected leaves and they give rise to, systemic acquired resistance—a state in which the plant shows enhanced resistance to subsequent challenges by microbial pathogens. It is also known at salicylic acid (SAY) plays an important role in the signal transduction pathway leading to systemic acquired resistance as infection of leaves by microbial pathogens causes an increase in the endogenous levels of salicylic acid followed by the induction of PR proteins.

The present invention relates to an alternative signal transduction pathway, the stimulation of which also appears to induce resistance to pathogens, especiallv microbial pathogens and which may be of use in addition to, or instead of, the salicylic acid pathway.

Previous work has demonstrated that exogenous application ofjasmonate and methyl jasmonate on potato and tomato plants induces resistance against the late blight fungus Phytophthora infestans (Cohen et al. 1993). There is, however, no teaching of the protection of a plant against a pathogen by inducing expression of a plant defensin gene.

Treatment with methyl jasmonate did not confer resistance in either of two other plant/pathogen systems. namely barleyl/Erysiphe graminis (Kogel et al. 1995) or ricel/Magnaporthe grisea (Schweizer et al. 1997).

It is well documented that compounds known to activate the salicylate-dependent pathogen-induced defence response, such as salicylic acid itself, acetylsalicylic acid, 2,6-dichloroisonicotinic acid (INA) and 1,2,3-benzothiadiazole-7-carbothioic acid S-methylester, can induce resistance to a range of microbial pathogens, including viruses, bacteria and fungi (White et al. 1979; Kessmann et al. 1994; Lawton et al. 1996; Friedrich et al.; Gorlach et al.). Interestingly, 1,2,3-benzothiadiazole-7-carbothioic acid S-methylester failed to protect tobacco plants against infection by the necrotrophic pathogens Botrvtis cinerea and Alterniaria alternata (Lawton et al.).

Three Arabidopsis genes have previously been identified. namely PR-1, PR-2 (β-3-glucanase) and PR-5 (osmotin-like protein), that are coordinately and systemically induced upon pathogen infection (Uknes et al., 1993; Dempsey et al., 1993; Mauch-Mani and Slusarenko, 1994). These genes are all highly induced upon exogenous application of SA or INA, a synthetic compound that appears to mimic the action of SA (Uknes et al., 1992; Cao et al., 1994).

The present inventors have discovered that the induction of genes which encode protective proteins is not always dependent upon the salicylic acid pathway but may be related to a quite separate pathway

According to a first aspect of the present invention, there is provided a method of protecting a plant against a pathogen, the method comprising inducing expression of a plant defensin gene by stimulating the jasmonate and/or ethylene pathways.

According to a second aspect of the present invention, there is provided a method of inducing expression of a plant defensin gene by applying to the plant one or more of ethylene, jasmonate, an agent which mimics the action of ethylene or jasmonate and an agent which causes oxidative stress.

According to a third aspect of the present invention, there is provided a composition which is capable of inducing expression of a plant defensin gene comprising one or more of jasmonic acid, a jasmonate, ethylene, an agent which mimics the action of ethylene or jasmonate and an agent which is capable of causing oxidative stress.

According to a fourth aspect of the present invention, there is provided composition which is capable of inducing expression of a plant defensin gene comprising one or more of an ethylene-generating compound, a lipid derived signal molecule, salicylic acid, functional analogues of salicylic acid and reactive oxygen-generating compounds.

According to a fifth aspect of the present invention, there is provided a method for screening compounds for resistance inducing (defensin-inducing) activity, the method comprising applying to a plant or part of a plant a compound suspected of giving such resistance and detecting the expression of a plant defensin gene or a coordinately expressed gene.

According to a sixth aspect of the present invention, there is provided a promoter which is capable of inducing the expression of a plant defensin gene comprising a region which is induced by jasmonic acid or an agent which mimics the action thereof and/or ethylene or an agent which mimics the action thereof.

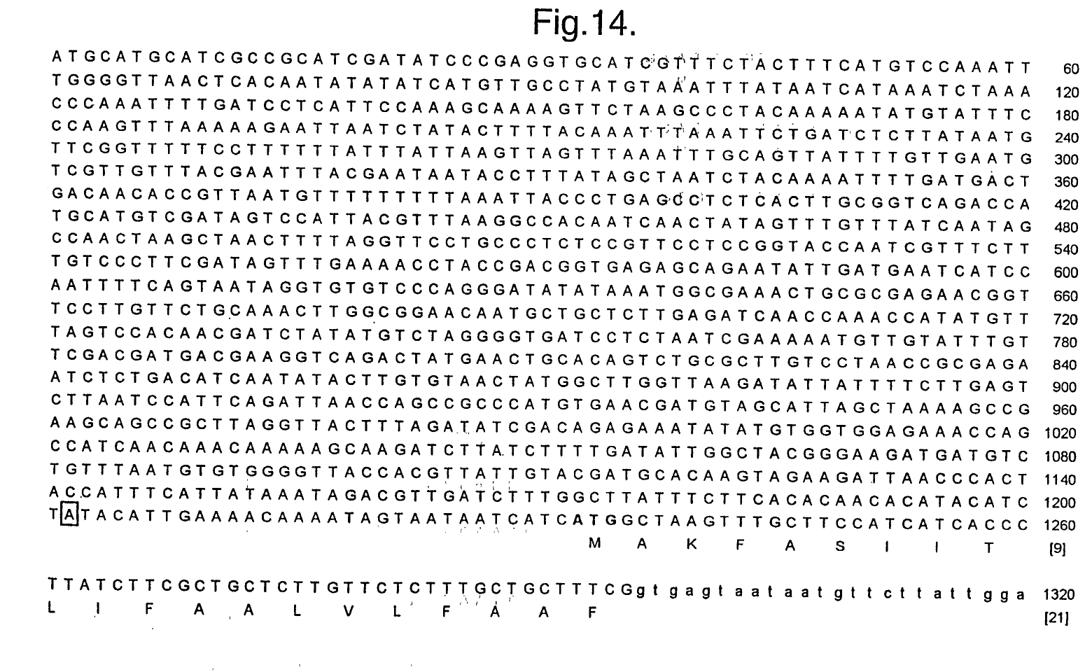

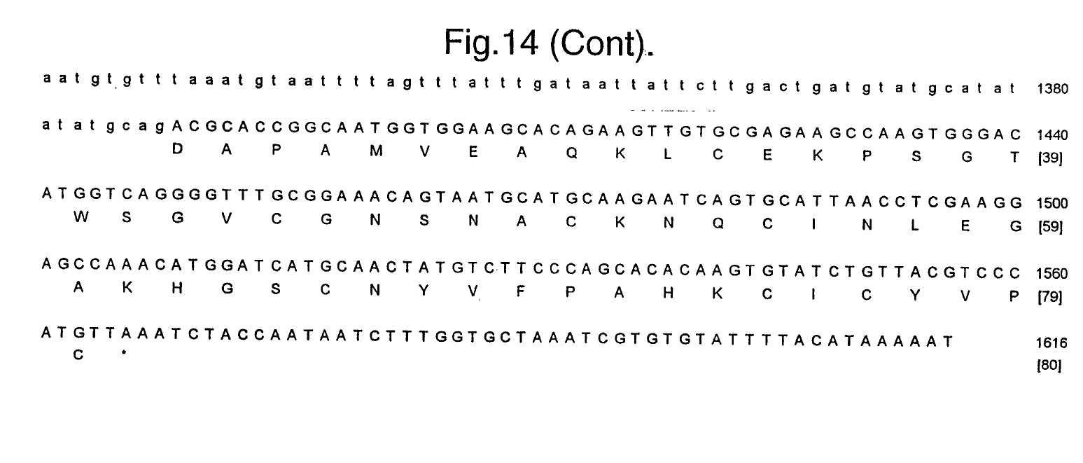

According to a seventh aspect of the present invention, there is provided a promoter region comprised within the nucleic acid sequence shown in FIG. 14, or a sequence that has substantial homology with that shown in FIG. 14, or a variant thereof.

Preferably, the plant defensin gene is induced by stimulating the jasmonate and ethylene pathways.

Preferably, the pathogen is a necrotrophic pathogen.

Preferably, the pathogen is a microbial pathogen.

Preferably, the pathogen is a fungus.

Preferably, the jasmonate and/or ethylene pathways are stimulated by the application of ethylene, jasmonic acid or ajasmonate. However, the application of an agent which mimics the action of ethylene or jasmonic acid would also be effective. Furthermore, it seems that expression of plant defensins can be induced by application of non-herbicidal amounts of agents which are capable of causing oxidative stress. Examples of such agents include diphenyl herbicides such as paraquat or diquat which result in the formation of superoxide anions or rose bengal which leads to the production of singlet oxygen species.

Preferably, the stimulation of the jasmonate and/ or ethylene pathways involves the signal transduction components EIN 2 and COI 1 from Arabidopsis. However, stimulation of these pathways may involve corresponding gene products in other plants which are substantially homologous to EIN 2 and COI 1.

Preferably, the ethylene-generating compound is selected from ethylene, ethephon and aminocyclopropanecarboxylic acid.

Preferably, the lipid-derived signal molecule is selected from arachidonic acid and derivatives thereof, linolenic acid and derivatives thereof and jasmonate and derivatives thereof.

Preferably, the reactive oxygen-generating compound is selected from paraquat. diquat, rose bengal and cosine.

Any plant species may be used but particularly suitable plants include radishes. tobacco or Arabidopsis species. When Arabidopsis species are used, the defensin may be the product of the plant defensin gene PDF 1.2 (FIG. 14), a sequence that has substantial homology with the sequence of PDF 1.2, or a variant thereof.

As mentioned above, compounds which mimic the acitivity of jasmonate or ethylene will also induce the expression of plant defensin genes and this means that the pathway can be used to screen for compounds activating endogenous defence mechanisms.

Although whole plants may be used in the screen, it may be more convenient to use parts of plants, particularly tissues such as the leaves.

The detection may be carred out by any-suitable means, for example by using antibodies agains the gene products of PDF 1.1, or PDF 1.2, on a related plant defensin or by means of a reporter gene such as a GUS gene or a luciferase gene linked to the promoter region of PDF 1.2.

An advantage of the method of the present invention is that it does not involve the use of cytotoxic or potentially harmful chemicals that directly interfere with living microbial cells but makes use of chemicals that activate existing defence mechanisms in plants.

An advantage of the composition of the present invention is that it can be used to give a plant resistance against certain types of pathogens for instance against necrotrophic pathogens. Alternatively, the composition of the present invention may be used to give plants protection against a broad spectrum of pathogens by using compounds which induce the salicylic, jasmonate and/or ethylene pathways.

A preferred embodiment of the present invention is a method of protecting a plant of the Arabidopsis species against a fungus, the method comprising inducing expression of a plant defensin gene by stimulating the jasmonate and/or ethylene pathways wherein expression of the defensin gene is induced by treatment of leaves with 0.5 μM methyl jasmonate in an atmosphere containing 25 ppm ethylene.

An even more preferred embodiment of the present invention is a composition which is capable of inducing expression of a plant defensin gene in order to protect a plant against attack by a number of pathogens, wherein the composition comprises ethylene, methyl jasmonate and salicylic acid. In this way, both the salicylate-dependent defence pathway and the jasmonate and/or ethylene pathways may be activated.

The term “variant thereof” with reference to the present invention means any substitution of, variation of, modification of, replacement of, deletion of or the addition of one or more nucleotides from or to the gene sequence providing product of the resultant sequence is capable of anti-pathogenic activity. The term also includes sequences that can substantially hybridise to the gene sequence. It also includes DNA which hybridises to the DNA of the present invention and which codes for at least part of the gene sequence. Preferably, such hybridisation occurs at, or between, low and high stringency conditions. In general terms, low stringency conditions can be defined as 3×SSC at ambient temperature to about 65° C., and high stringency conditions a 0.1×SSC at about 65° C. SSC is the name of a buffer of 0.15 M NaCl, 0.015 M trisodium citrate. 3×SSC is three times as concentrated as SSC and so on.

The term “substantial homology” covers homology with respect to at least the essential nucleotide/s of the gene sequence providing the homologous sequence acts as a defensin gene ie its product is capable of giving resistance against a pathogen of a plant. Typically, homology is shown when 60% or more of the nucleotides are common with the gene sequence of the present invention, more typically 65% preferably 70% more preferably 75%, even more preferably 80% or 855 and. especially preferred. are 90%, 95%, 98% or 99% or more homology.

The term “microbial” includes bacteria, fungi and viruses.

Qne of the genes that is activated in Arabidopsis upon microbial attack and whose expression is not dependent on salicylic acid but rather requires functional components of the ethylene and jasmonic acid response pathways is a plant defensin gene. Plant defensins are a family of cysteine-rich basic proteins of about 5 kDa in length which are structurally related to the antimicrobial insect defensins found in various insect species-(Broekaert et al-Plant, 1995). A number of these plant defensins were known to be potent inhibitors of fungal growth which suggested that they play a role in host defence. It has been shown that expression of a plant defensin gene from radish in transgenic tobacco plants confers resistance to the fungal pathogen Alternania longipes (Terras et al., 1995). The pathogen induced plant defensin gene is most probably not the only gene activated via the jasmonate and/or ethylene-dependent pathways ie a salicylic acid independent pathway, but it can be used as an indicator to monitor the activation of this pathway. However, any other gene operating in the same pathway could be used for the same purpose.

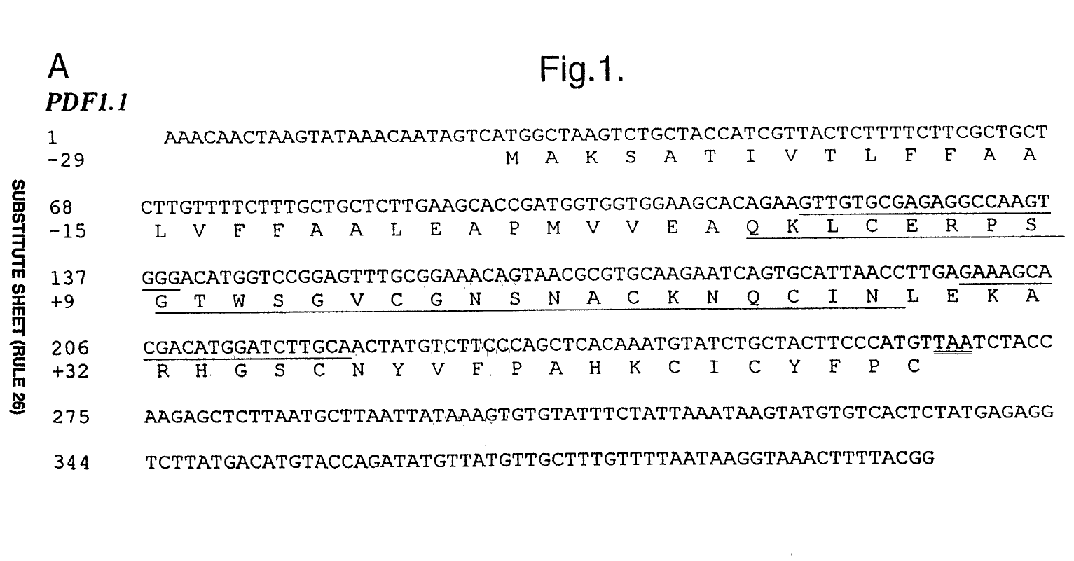

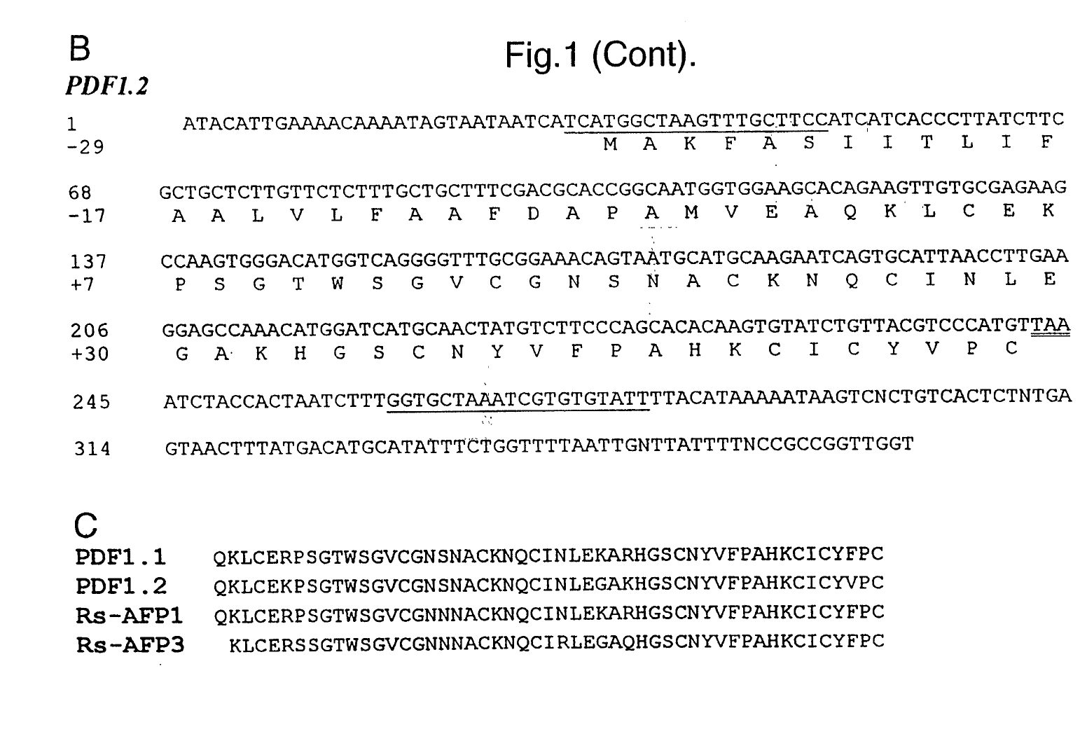

As described in greater detail in the examples below, two Arabidopsis genes encoding plant defensins were found to be expressed. These genes were found in a search of the Genbank database for sequences homologous to the nucleotide sequence for the cDNA of Rs-AFP1, a known radish plant defensin, and have the Genbank accesssion numbers Z27258 and T04323 and were designated PDF1.1 and PDF1.2 respectively. Their sequences are given in FIG. 1A and 1B and as SEQ ID NOs 1 and 2. These sequences were identified as expressed sequence tags in CDNA libraries prepared either from dry seed mRNA (PDF1.1) or seedling mRNA (PDF1.2). The genes encode preproteins with a putative peptide signal and a mature plant defensin domrnain which are 98% and 92% identical to the radish protein.

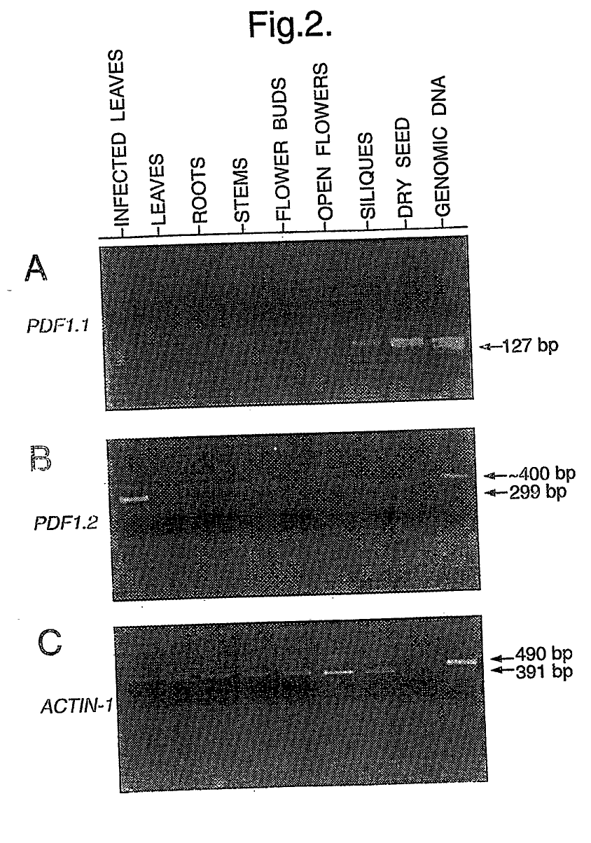

The expression patterns of PDF1.1 and PDF1.2 were analysed by reverse transcription polymerase chain reaction (RT-PCR) on DNAase-treated RNA isolated from different Arabidopsis organs (FIG. 2). As a control for the RT-PCR reactions, a primer pair was designed for amplification of sequences corresponding to the region of the Arabidopsis ACTIN-1 gene (Nairn et al, Gene 1988) encompassing a 99 base pair intron. In this way, products derived from PCR amplification of genomic DNA can be discriminated by size from true RT-PCR products obtained from RNA. As can be seen in FIG. 2, RNA samples from all analysed tissues, except dry seed, yielded ACTIN-1 RT-PCR amplification products of the expected size, whereas genomic DNA yielded aPCR product which was about 100 bp longer. RT-PCR with a primer pair specific for PDF1.1 showed amplification products with RNA from siliques and dry seed as templates. The primer pair specific for PDF1.2 did not yield RT-PCR amplification products in any tisssue analysed from healthy plants. However, amplification products of the expected size were detected upon RT-PCR performed on RNA from leaves infected with Alternaria brassicicola strain MUCL 20297, a fungus causing brown necrotic lesions which do not spread over time. The PCR amplification product obtained with genomic DNA was about 100 bp longer than that obtained by RT-PCR on RNA from infected leaves, indicating that the PDF1.2-specific primers span an intron in the PDF1.2 gene. This was not observed with PDF1.1-specific primers.

It was concluded that Arabidopsis contains two genes which encode highly homologous plant defensins but which have a totally different expression pattern. PDF1.1 is expressed predominantly in seed and is considered to be the homologue of the radish Rs-AFP1 and RsAFP2 genes. whereas PDF1.2 is expressed in leaves upon pathogen stress and can be considered as the homologue of the RsAFP3 and RsAFP4 genes from radish (Terras et al., 1 995).

The PDF1.1 and PDF1.2 genes and their expression products were used in the further investigations which are described in more detail in the examples below.

Analysis of the expression of two plant defensin genes identified via their expressed sequence tag (EST) sequences revealed that they are differentially expressed and only one of these tnvo genes. designated PDF1.2 , appears to be pathogen-inducible. Although only two homologous plant defensin EST sequences have been identified so far, it is possible that other plant defensin genes may be present and possibly expressed in Arabidopsis because Southern blot analysis of Arabidopsis genomic DNA digested with four independent restriction enzymes and using a cDNA clone for Rs-AFP2 as hybridisation probe revealed 3 to 5 bands depending on the enzyme used (data not shown). At present the expression pattern of other putative defensin genes during pathogenesis remains unknown and will require a careful examination of the gene family.

In the present work we have found that the expression of plant defensin genes. including at least PDF1.2 , is also induced systemically upon, pathogen infection but this induction follows a response pathway that is clerly diffetent from that followed by PR-proteins. This finding is based on several lines of evidence.

First, Arabidopsis plant defensins were not induced upon external application of either SA or INA. In contrast, treatments with either methyl jasmonate, ethylene or the reactive oxygen species generating compounds paraquat and rose bengal caused accumulation of plant defensin transcripts. Second, the pathogen-induced systemic expression of plant defensin genes was not reduced in the npr-1 Arabidopsis mutant which is known to be blocked in the response pathway leading from SA to actuvation of PR-protein genes (Cao et al., 1994). Third, plant defensin genes were not constitutively expressed in the cpr1 Arabidopsis mutant which displays constitutively elevated levels of endogenous SA as well as of PR-1, PR-2 and PR-5 transcripts (Bowling et al., 1994). Fourth, analysis of the etr1-3, ein2 and coil mutants of,Arabidopsis demonstrated that the mechanism leading to induction of plant defensins in response to fungal challenge appears to involve components from or shared by the ethylene and jasmonate response pathways. These results therefore indicate that two separate classes of antifungal proteins (exemplified by PR-1 and plant defensin respectively) can be induced both locally and systemically by distinct signalling processes.

The ein2 Arabidopsis mutant. identified by a lack of morphological response when grown in the presence of ethylene (Guzman and Ecker, 1990) is virtually blocked in its pathogen-induced expression of plant defensin genes both in pathogen-treated leaves and in non-treated, systemic leaves. In contrast, the etr1-3 mutant, which is a partially ethylene-insensitive mutant (Chang et al., 1993), had a normal plant defensin response in infected leaves but exhibits reduced but not abolished plant defensin gene expression in systemic leaves of infected plants-The incomplete suppression of plant defensin gene expression is pathogen-challenged etr1-3 plants is most probably due to the leakiness of this particular mutant allele. The coil mutant is known to be less sensitive than wild-type plants to inhibition of root growth upon treatment with methyl jasmonate or coronatine, a bacterial phytotoxin acting as a jasmonate analog (Feys et al., 1994). This mutant showed an almost completely blocked pathogen-induced plant defensin response both in tie pathogen-treated and non-treated, systemic leaves.

From these analyses it thus appears that EIN2 and COI1 are required for local as well as systemic plant defensin induction, whereas ETR 1 appears to be involved only in the systemic response. Of these three genes, only ETR 1 has been identified. ETR 1 encodes a protein resembling bacterial two-component histidine kinase sensors, and genetic and biochemical evidence indicates that ETR 1 is an ethylene receptor (Schaller and Bleecker, 1995). The gene product EIN2 acts downstream of ETR 1 in the ethylene response pathway (Ecker, 1995). COI1 has not been characterized to date but it is believed to be involved in signal transduction initiated by jasmonates (Feys et al., 1994).

Interestingly, ein2 plants have previously been found to display decreased chlorotic lesion formation relative to wild-type plants when infiltrated with virulent strains of the bacterium Pseudomonas syringae pv. tomato (Bent et al., 1992). Mutants carrying the etr1-3 mutation (previously called ein1-1) responded like wild-type plants. Although ein2 plants showed decreased disease symptoms relative to wild-type and etr1-3 plants the P. syirngae pv. tomato bacteria multiplied equally well in these three genotypes.

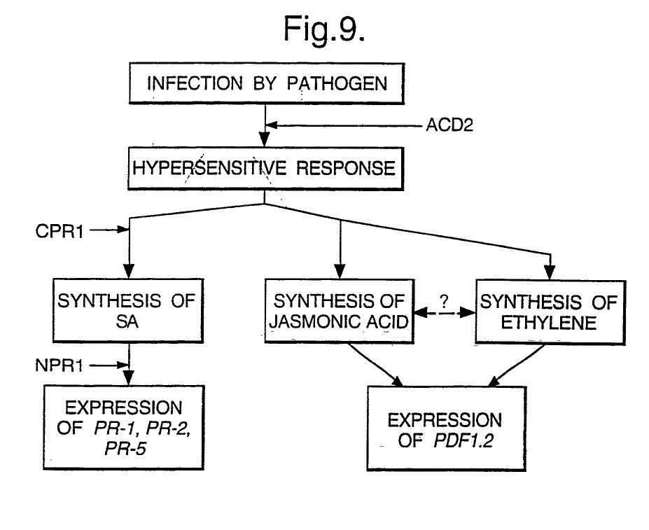

A model for two separate pathways leading to induction of defence-related genes upon pathogen stress is presented in FIG. 9. In this model, the hypersensitive response is positioned above the bifurcation point, because acd2 Arabidopsis mutants developing spontaneous hypersensitive response-like lesions (Greenberg et al., 1994) were found to contain enhanced transcript levels of both plant defensins and PR-protein (Greenberg et al. 1994). The pathway leading to PR-protein gene expression via salicylic acid involves the signal transduction components NPR1 and CPR1, whereas the pathway leading to plant defensin gene expression would require EIN2 and COI1. There may also be some degree of “cross-talk” between both defence response pathways and it appears that inhibition of SA accumulation results in enhanced activation of the jasmonate and/ or ethylene pathways ie SA-independent pathway. In this regard, acetylsalicylic acid was shown to inhibit jasmonate biosynthesis (Peña-Cortes et al., 1993), whereas SA was found to inhibit the induction of proteinase inhibitors by jasmonic acid (Doares et al., 1995).

Other defence-related genes may be activated coordinately with plant defensins via the SA-independent pathway depicted above. A first likely candidate is Hel, a hevein-like (PR-4-like) gene that is induced both locally and systemically-upon viral infection (Potter et al., 1993). Hel is strongly induced by ethylene but-only weakly by SA; whereas PR-1, PR-2 and PR-5 are not ethylene-inducible but-strongly SA-inducible (Potter et al., 1993). A second candidate is the thionin gene Thi2.1 which is induced upon fungal infection and methyl jasmonate treatment, but not upon SA treatme nt (Epple et al., 1995). A third possible. candidate is the basic chitinase gene CHIT-B which is induced upon ethylene treatment in wild-type plants but not in ein2 or etr1-3 mutants (Chen and Bleecker, 1995). It was observed that the induction of CHIT-B in virus-infected leaves of Arabidopsis followed different kinetics relative to the induction of PR-1, PR-2 and PR-5 (Dempsey et al., 1993).

There is also evidence for the existence of two separate pathways for pathogen-inducible responses in tobacco. The first pathway leads to induction of acidic PR-protein genes such as PR-1, PR-2, PR-3 (acidic chitinase), PR-4 and PR-5, whereas the second pathway results in induced expression of the basic β-1,3-glucanase and basic chitinase genes. The first group of genes is strongly activated by SA, while the second group responds to ethylene (Meins et al., 1991). Interestingly, transgenic tobacco plants expressing a gene encoding the Al subunit of cholera toxin, a G-protein inhibitor, showed constitutive expression of the acidic PR-protein genes but not of the basic β-1,3-glucanase and basic chitinase genes (Beffa et al., 1995). The acidic PR-protein genes are induced both locally and systemically upon challenge with tobacco mosaic Virus (TMV) (Ward et al., 1991; Brederode et al., 1991). The basic β-1,3-glucanase and basic chitinase genes, on the other hand, are also strongly induced in the inoculated leaves but there is contradictory evidence as for their systemic inducibility. Based on RNA blot analysis, no significantly enhanced transcript levels of these genes could be detected in uninoculated leaves of TMV-infected plants (Ward et al., 1991; Brederode et al., 1991), whereas the corresponding proteins were found to accumulate in such leaves based on Western blot analyses (Heitz et al., 1994).

It may be speculated that pathogen-induced expression of the basic β-1,3-glucanase and basic chitinase genes in tobacco follows a pathway equivalent to that leading to induced plant defensin gene expression in Arabidopsis. One apparent difference is that induced expression of plant defensins occurs in systemic leaves both at the transcript level and at the protein level. It should be taken into account that Arabidopsis is much smaller than tobacco and hence that systemic responses in the former are measured over much shorter distances. Another obvious difference between basic β-1,3-glucanase and basic chitinase expression in tobacco on the one hand. and plant defensin gene expression in Arabidopsis on the other hand, is that the tobacco genes are wound-inducible (Brederode et aL, 1991), whereas plant deifensin genes in Arabidopsis are not.

We have demonstrated that a pathogen-induced protein cross-reacting with antibodies raised against a PDF1.2 analog from radish (Rs-AFP1) possesses strong antifungal activity in vitro. This protein accumulates to rather high levels upon fungal infection, namely up to 2% and 1% of total proteins in inoculated and untreated, systemic leaves, respectively. We have previously shown that transgenic tobacco plants producing a PDF1.2 analog from radish (Rs-AFP2) at about 0.25% of total soluble proteins are more resistant to A. longipes than untransformed plants (Terras et al., 1995). Based on these observations it is likely that plant defensins have a role to play in host defence. There is now compelling evidence that a SA-dependent pathway is important for mounting resistance to some pathogens, including P. syringae pv. tomato and Peronospora parasitica. Indeed, nahG-expressing plants were found to be considerably more sensitive to these pathogens relative to wild-type plants (Delaney et al., 1994) and, unlike wild-type plants, were unable to mount systemic acquired resistance (Lawton et al., 1995). Evidence is presented here to show that the SA-independent pathway (leading to activation of plant defensin genes. and possibly other defence-related genes as well) also contribute to resistance, albeit to different types of pathogens. Indeed, we have shown that the ein 2 and coi 1 mutants, which are apparently blocked in the SA-independent pathway, are more susceptible to infection by Botrytis cinerea than wild type plants.

The invention will now be further described only by way of example in the ofllowing examples and in the Figures in which:

FIG. 1 shows nucleotide sequences and deduced amino acid sequences of expressed sequence tags Z27258 and T04323 corresponding to PDF1.2 and PDF1.2, respectively.

(A) Nucleotide sequence and deduced amino acid sequence of Z27258 corresponding to PDF1.1 . The experimentally determined N-terminal region of PDF1.1 previously called At-AFP1 (Terras et aL, 1993) is underlined. The nucleotide at position 123 (T in Z27258) has been replaced by a G according to additional sequence data.

(B) Nucleotide sequence and deduced amino acid sequence of T04323 corresponding to PDF1.2.

(C) Alignment of the amino acid sequences of mature-Rs-AFP1 and Rs-AFP3 from radish seed and leaves, respectively (Terras et al., 1995; F.R.G. Terras, unpublished results) with the mature domain of PDF1.1 and the putative mature domain of PDF1.2.

Underlined nucleotide sequences in (A) and (B) correspond to the positions of primers used for RT-PCR. Stop codons in (A) and (B) are double underlined.

FIG. 2 shows the expression pattern of PDF1.1 and PDF1.2 in Arabidopsis;

(A) RT-PCR analysis using a primer pair specific for PDF 1.1.

(B) RT-PCR analysis using a primer pair specific for PDF 1.2.

(C) RT-PCR analysis using a primer pair specific for ACTIN-1.

RT-PCR reactions were performed on DNase-free total RNA isolated from different Arabidopsis organs. Roots, stems, flower buds, open flowers and siliques were collected from 7-week-old flowering plants. Leaves and infected leaves were collected from 4-week-old plants. Infected leaves were inoculated with A. brassicicola and collected after 3 days of incubation.

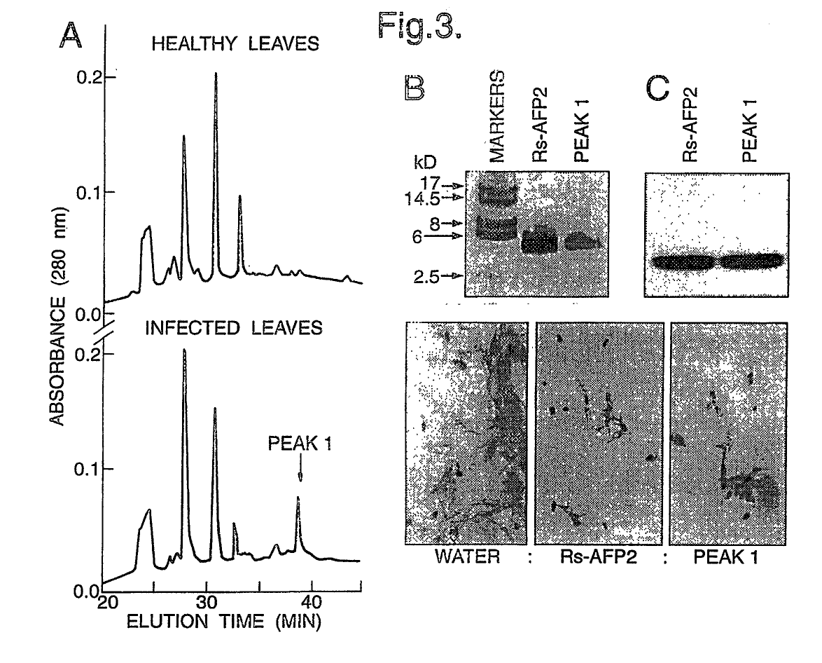

FIG. 3 shows purification and characterisation of a plant defensin from infected Arabidopsis leaves;

(A) Separation of the basic protein fraction of healthy Arabidopsis leaves (upper part) and A. brassicicola-infected Arabidopsis leaves (lower part) on a C2/C 18 silica reversed-phase chromatography column. The column was eluted with a linear gradient from 0% to 50% (v/v) of acetonitrile in 0.1% (v/v) trifluoroacetic acid.

(B) Electrophoretic analysis of proteins contained in peak 1. Two hundred ng of protein contained in-peak 1 and 200 ng of purified Rs-AFP2 were electrophoresed on an SDS-PAGE gel (Phastgel High Density, Pharmacia) and silver stained. Sizes of the molecular mass markers are indicated at left in kilodaltons.

(C) Immunoblot of proteins contained in peak 1 after SDS-PAGE using anti-Rs-AFP1 antibodies. Lanes were loaded with either 200 ng of purified Rs-AFP2 or 200 ng of purified protein contained in peak 1.

(D) In vitro antifungal activity against A. brassicicola of water (negative control, left panel), purified Rs-AFP2 at 5 μig/mL (middle panel) or 5 μg/mL purified protein contained in peak 1 (right panel). Micrographs were taken after 24h of incubation at 22° C.

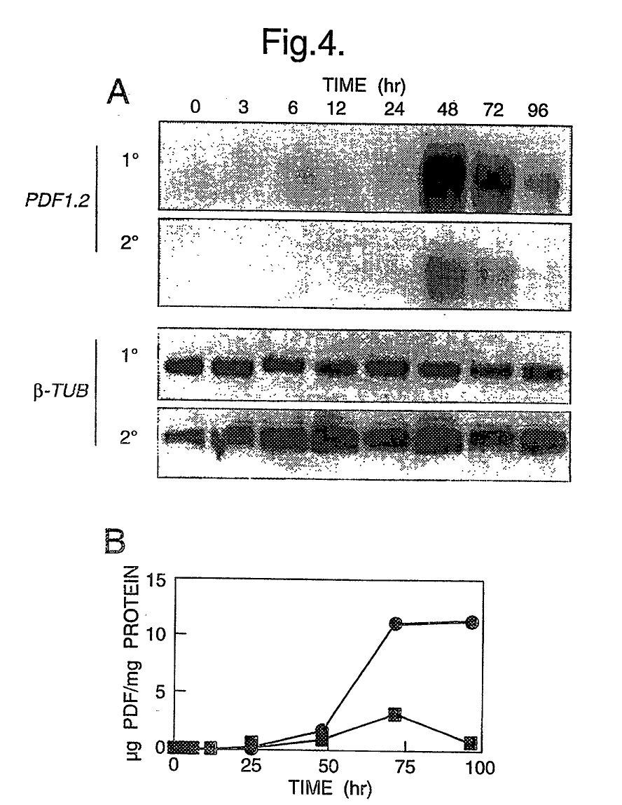

FIG. 4 shows expression of plant defensins in Arabidopsis upon fungal infection;

(A) RNA gel blot analysis of PDF1.2 and β-Tubulin (β-TUB) expression in pathogen-treated leaves (1°) and non-treated, systemic leaves (2°) of infected plants. All analyzed samples represent 4 μg of total RNA.

(B) Plant defensin (PDF) content in pathogen-treated leaves (circles) and non-treated, systemic leaves (squares) of infected plants as determined by ELISA using antigen affinity-purified anti-Rs-AFP1 antiserum. Values are means (±standard error) from three independent determinations. Total RNA and proteins were isolated from pathogen-treated and non-treated, systemic Arabidopsis leaves collected 0h, 3h, 6h, 12h, 24h, 48h, 72h and 96h after inoculation with 5 μL drops (5 drops per leaf) of A. brassicicola spores at 5×10⁵ spores/mL.

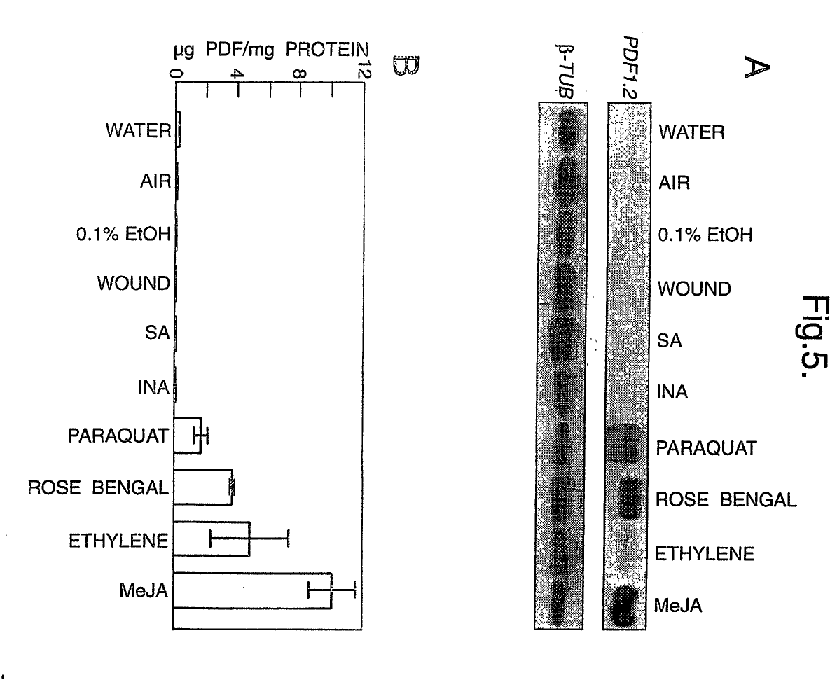

FIG. 5 shows induction of plant defensins in Arabidopsis leaves upon chemical treatments and wounding;

(A) RNA gel blot analysis of PDF1.2 and β-Tubulin (β-TUB) expression in leaves wounded or treated with the indicated chemicals. All analyzed samples represent 4 μg of total RNA.

(B) Plant Defensin (PDF) content as determined by ELISA using antigen affinity-purified anti-Rs-AFP 1 antiserum. Values are means (±standard error) of three independent determinations. Arabidopsis leaves were inoculated with 5 μL drops (5 drops per leaf) of water, SA (5 mM), INA (1 mg/mL), paraquat (25 μM), rose bengal (20 mM), methyl jasmonate (45 μM in 0.1% (v/v) ethanol) or 0.1% (v/v) ethanol (0.1% EtOH). Ethylene treatment was performed by placing plants in an air-tight chamber with an ethylene concentration of 20 ppm. Control plants (air) were incubated in an identical chamber without ethylene. Wounding was applied by making incisions in the leaf with a scalpel. All leaf samples were collected 48h after initiations of the treatments. The experiment was repeated once with similar results.

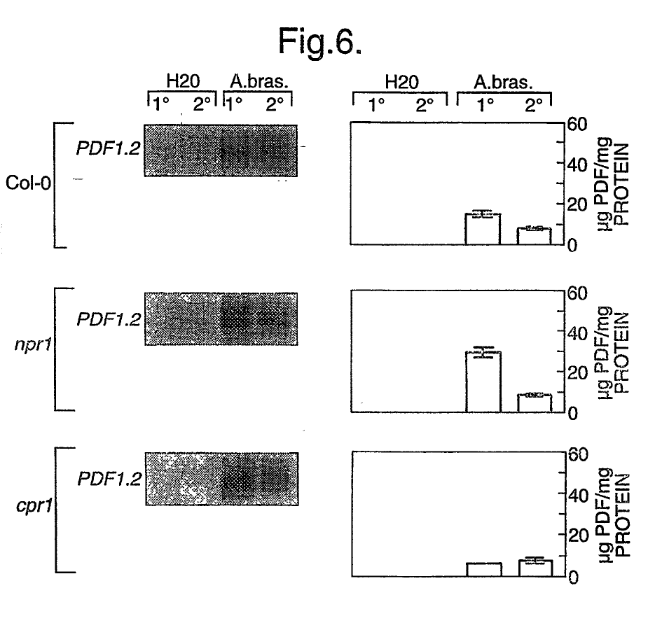

FIG. 6 shows induction of plant defensins in Arabidopsis wild-type (Col-0), and in Arabidopsis mutants (npr1 and cpr1) affected in the SA-signalling pathway;

The left part of the figure shows RNA gel blot analyses of PDF1.2 expression. The samples represent 4 μg of total RNA.

The right part shows plant defensin (PDF) contents as determined by ELISA using antigen affinity-purified anti-Rs-AFP1 antiserum. Values are means (±standard error) of three independent determinations.

Arabidopsis plants were- inoculated with A. brassicicola (A. bras.) by applying 5 μL drops of a spore suspension (5×10⁵ spores/mL) on four lower rosette leaves (5 drops per leaf). Control plants were treated identically with 5 μL drops of water (H₂O). Pathogen-treated leaves (1°) and non-treated leaves of the same -plants (2°) were collected 3 days after inoculation. Total RNA and proteins were extracted as described (see methods). The experiment was repeated twice with similar results.

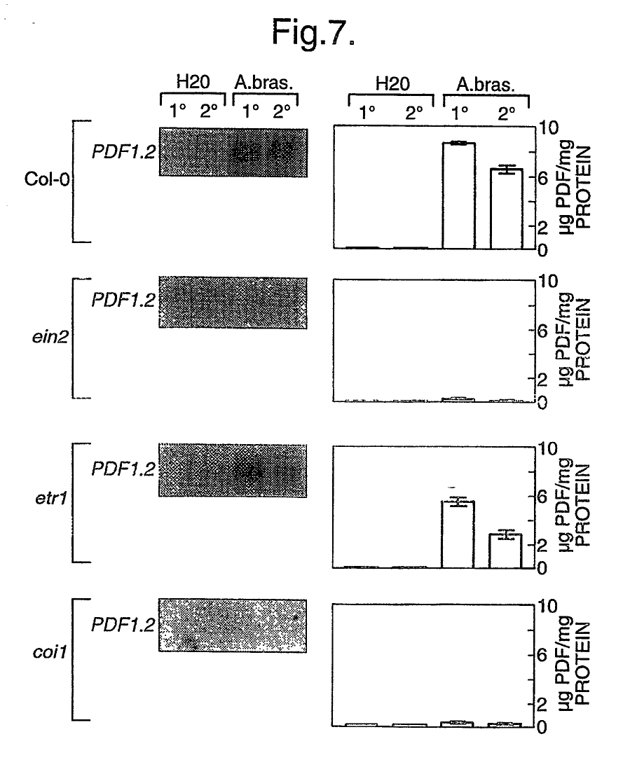

FIG. 7 shows induction of plant defensins in Arabidopsis wild-type (col-0), in Arabidopsis mutants (ein2 and etr1-3) affected in the ethylene response pathway and in a mutant (coil) affected in the jasmonate response pathway.

The left part of the figure shows RNA gel blot analyses of PDF1.2 expression. The samples represent 4 μg of total RNA.

The right part shows plant defensin (PDF) contents as determined by ELISA using antigen affinity-purified anti-Rs-AFP1 antiserum. Values are means (±standard error) of three independent determinations.

Arabidopsis plants were inoculated with A. brassicicola (A. bras.) by applying 5 μL drops of a spore suspension (5×10⁵ spores/mL) on four lower rosette leaves (5 drops per leaf). Control plants were treated identically with 5 μL drops of water (H₂O) Pathogen-treated leaves (1°) and non-treated leaves of the same plants (2°) were collected 3 days after inoculation. Total RNA and proteins were extracted as described (see methods). The experiment was repeated twice with similar results.

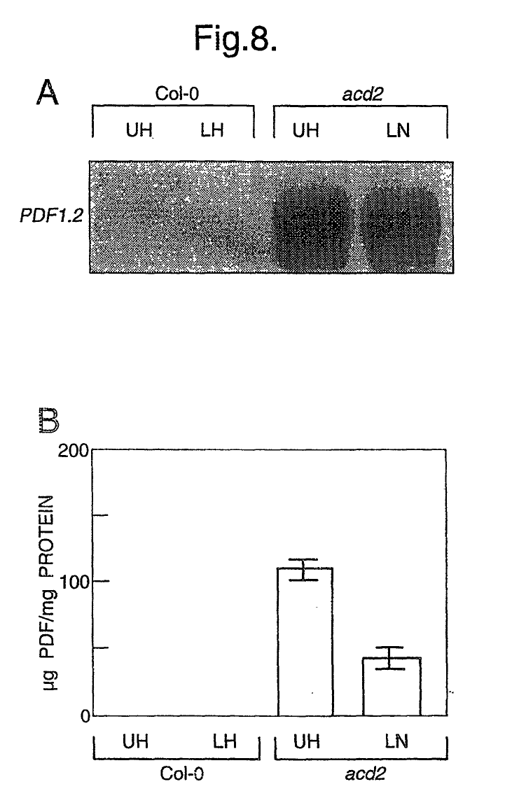

FIG. 8 shows induction of plant defensins in Arabidopsis wild-type (Col-0) and in anArabidopsis lesion mimic mutant (acd2).

(A) RNA gel blot analysis of PDF1.2 expression. All analyzed samples represent 4 μg of total RNA.

(B) Plant Defensin (PDF) content as determined by ELISA using antigen affinity-purified anti-Rs-AFP1 antiserum. Values are means (±standard error) of three independent determinations.

Total RNA and proteins were isolated from healthy asymptomatic upper rosette leaves (UH) and lower rosette leaves displaying -necrotic lesions (LN) collected from 5-week-old acd2-plants. as well as from healthy upper rosette (UH) and lower rosette leaves (LH) from control plants (Col-O) grown under identical conditions. The experiment was repeated twice with similar results.

FIG. 9 is a proposed model for the induction of defence-related genes via two separate pathways, namely a salicylate-dependent pathway and a jasmonate and/or ethylene-dependent pathways.

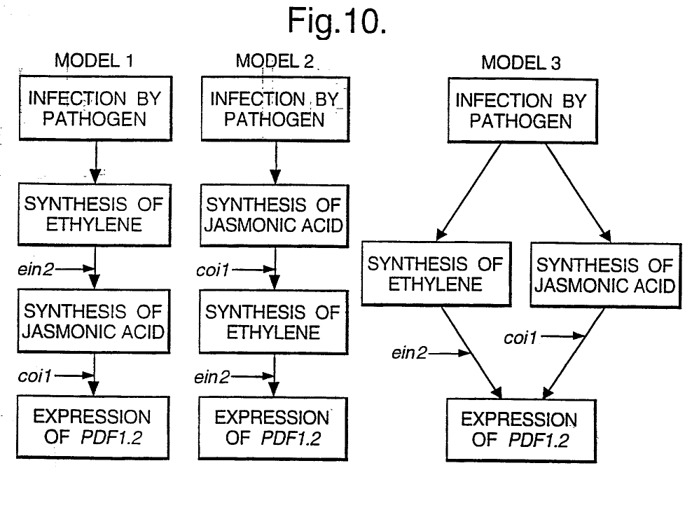

FIG. 10 shows three alternative models for the interaction between ethylene and jasmonate signals during activation of the PDF1.2 gene in pathogen-stressed Arabidopsis plants.

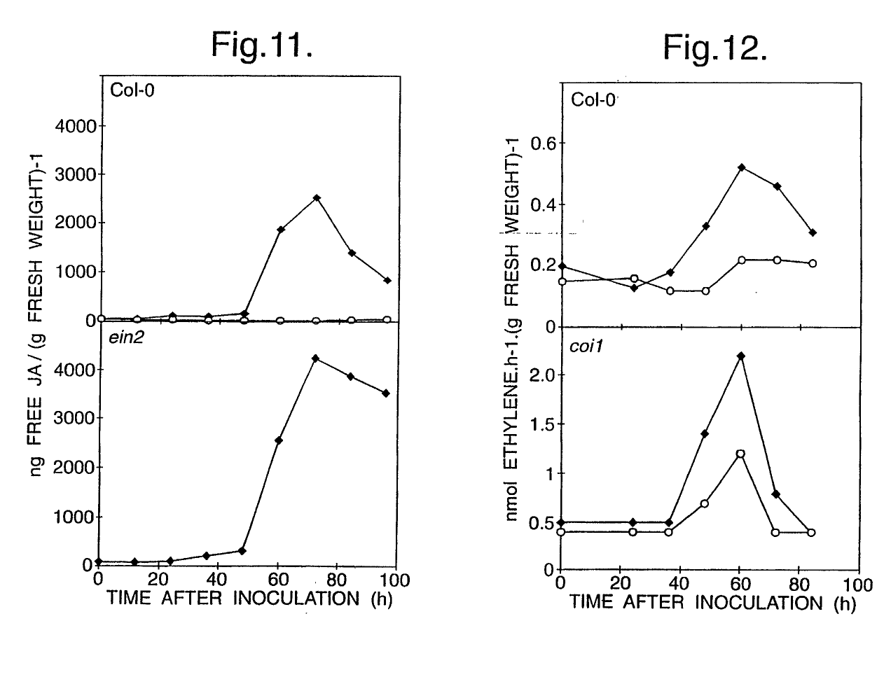

FIG. 11 shows a time course ofjasmonic acid levels in Arabidopsis wild-type plants (Col-0, upper panel) and ethylene-insensitive mutants (ein2, lower panel) upon inoculation with Alternaria brassicicola (closed symbols) or mock inoculation with water (open symbols). Each datapoint is the average of two separate measurements on two sets of three plants each.

FIG. 12 shows a time course of ethylene production levels in Arabidopsis wild-type plants (Col-0, upper panel) and jasmonate-insensitive mutants (coil, lower panel) upon inoculation with Alternaria brassicicola (closed symbols) or mock inoculation with water (open symbols). Data for Col-0 are averages of three independent experiments with two plants for each time point. Data for coil are from a single experiment with two plants for each time point.

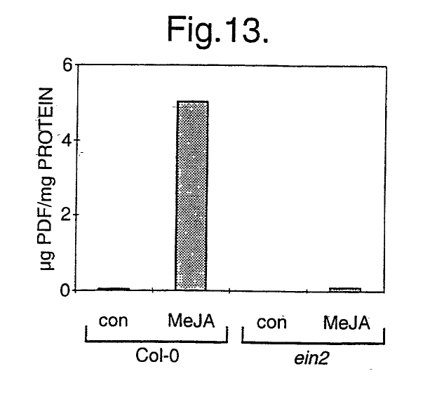

FIG. 13 shows the induction of plant defensins (PDF) in Arabidopsis,wild type plants (Col-0) and ethylene-insensitive mutants (ein2) upon treatment with 0.1% ethanol (con) or 50 μM methyl jasmonate in 0.1% ethanol (MeJA).

FIG. 14 shows the nucleotide sequence of the Arabidopsis PDF1.2 gene. The boxed nucleotide represents the first nucleotide of expressed sequence tag T04323. The amino acids of the gene product are shown below the corresponding codons of the coding region. The intron is shown in lower case letters.

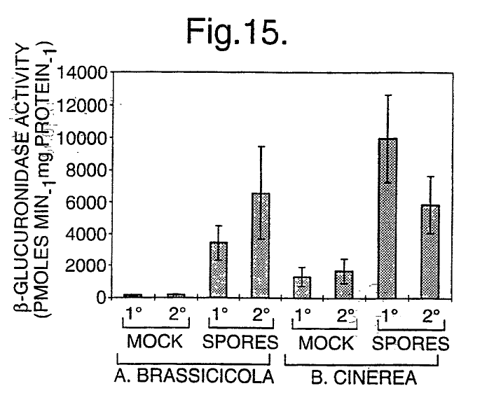

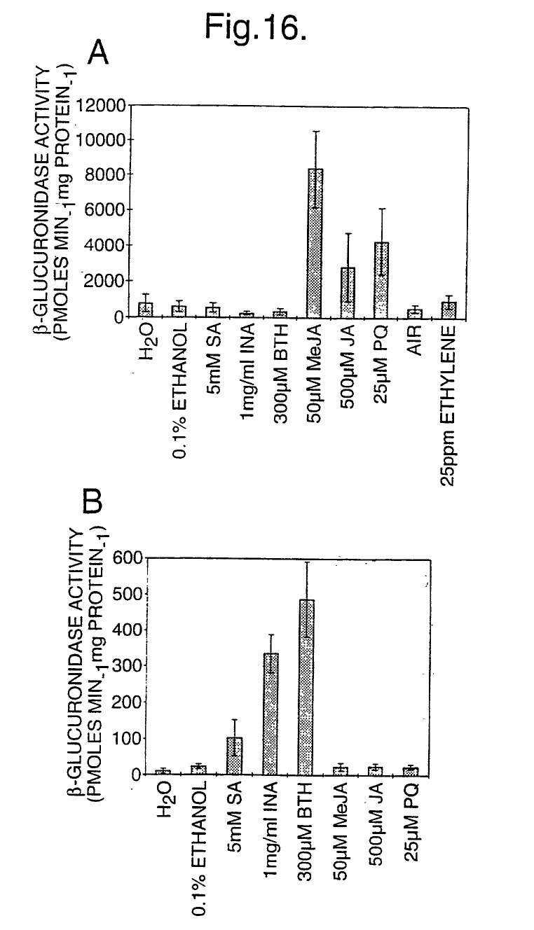

FIG. 15 shows β-glucuronidase activity in transgenic pPDF1.2-GUS-tNOS Arabidopsis plants upon inoculation with A. brassicicola (mock or spore-inoculated) or B. cinerea (mock or spore-inoculated). Treated leaves (1°) and non-treated leaves (2°) of the same plant were collected 3 days after treatmenf. Results are exptessed as averages±standard errors of four sets of two plants. FIG. 16 shows β-glucuronidase activity in transgenic pPDF1.2-GUS-tNOS Arabidopsis plants (panel A) and transgenic pBg12-GUS-tNOS Arabidopsis plants (panel B) upon treatment with various chemicals. Samples of treated leaves were collected 48 h after treatment. Results are expressed as averages±standard errors of four-inidvidually harvested plants.

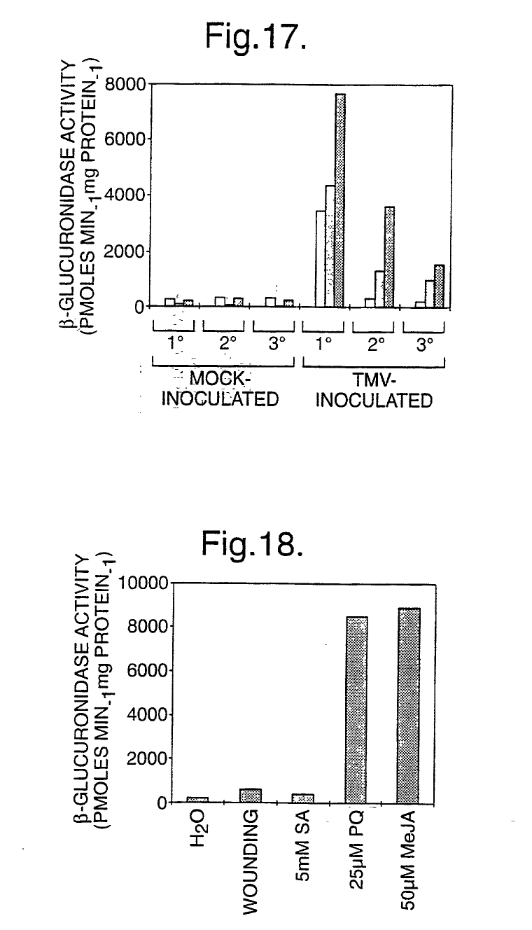

FIG. 17 shows β-glucuronidase activity in trarsgenic pPDF1.2-GUS-tNOS tobacco (cv. Xanthi-nc) plants with tobacco mosaic virus or mock-inoculated. The leaves just below the youngest fully expanded leaves were either virus- or mock-inoculated. Those leaves (1°), the youngest fully expanded leaves (2°) and the leaves just above the youngest fully expanded leaves (3°) were harvested separately at two days (black bars), 4 days (light grey bars) or 6 days (dark grey bars) following treatment.

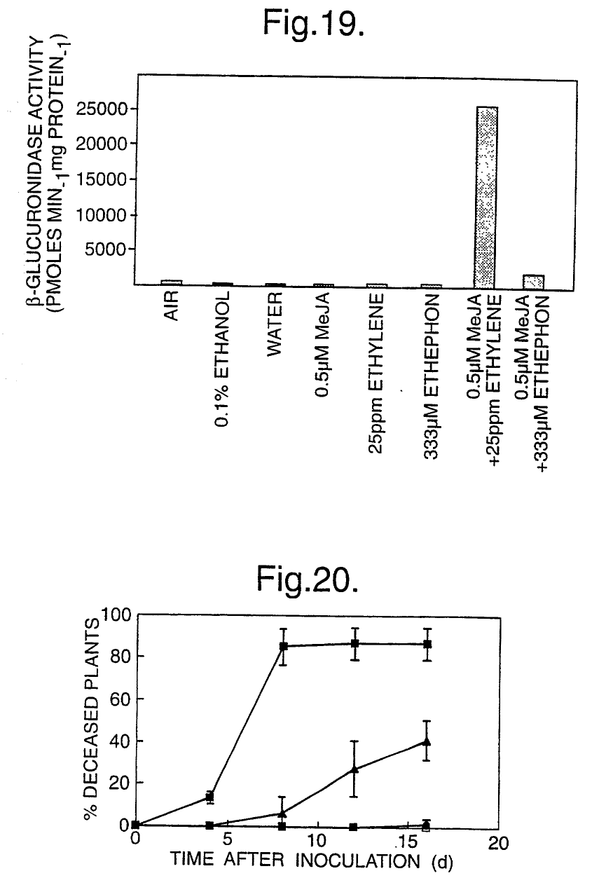

FIG. 18 shows β-glucuronidase activity in transgenic pPDF1.2-GUS-tNOS tobacco (cv. Xanthi-nc) plants upon wounding or treatment with various chemicals. Samples of treated leaves were harvested 48 h after treatment. FIG. 19 shows β-glucuronidase activity in transgenic pPDF1.2-GUS-tNOS Arabidopsis plants upon exposure to 25 ppm ethylene, treatment with 0.5 μM methyl jasmonate (MeJA, in 0.1% ethanol), 333 μM ethephon, 25 ppm ethylene plus 0.5 μM methyl jasmonate, 333 μM ethephon plus 0.5 μM methyl jasmonate, and the appropriate control treatments: air exposure and treatment with water and 0.1% ethanol.

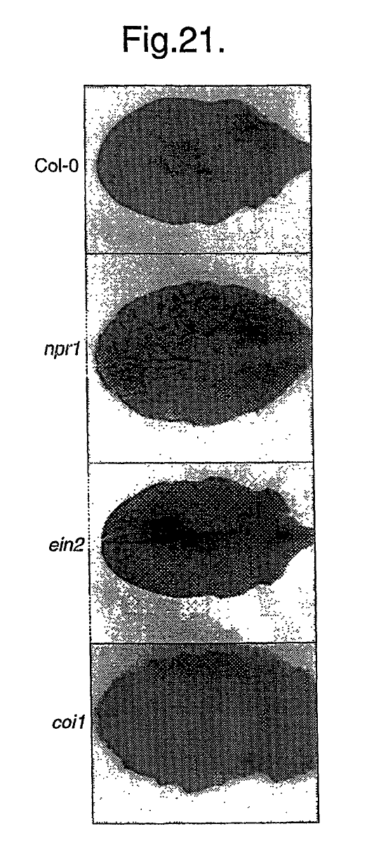

FIG. 20 shows the decay-ofArabidopsis wild type plants (Col-0, circles), ethylene insensitive mutants (ein2 triangles) and jasmonate insensitive mutants (coil. squares) after inoculation with the fungal pathogen Botrytis cinerea. Data represent averages with standard deviations of four independ ent experiments (Col-0 and ein2) and three independent expent ments (coil) preformed with series of 20 plants for each plant line.

FIG. 21 shows lactophenol/trypan blue staining of hyphal structures of the flungus Peronosporaparasitica pathovar Wela in leaves of inoculated Arabidopsis wild type plants (Col-0) and ein2, coil and nprl mutants.

### METHODS

Biological Material

The mutants npr 1 (Cao et al., 1994) and cpr 1 (Bowling et al., 1994) were provided by Dr. X. Dong (Duke University, Durham, N.C., USA). The ethylene response mutants ein2 (Guzrnan and Ecker, 1990) and etr1-3 (Bleecker et al.,1988; Chang et al., 1993) were obtained from the Arabidopsis Biological Resource Centre, Ohio, USA (accession number CS3071 and CS3070, respectively) and the lesion mimic mutant acd2 (Greenberg et al., 1994) was provided by Dr. F. Ausubel (Massachusetts General Hospital, Boston, USA). The jasmonate response mutant coil (Feys et al., 1994) was obtained from Dr. J. Turner:. (University of East Anglia, Norwich, UK). Since this mutation is recessive and causes male sterility, coil mutants were identified a posteriori in F2 plants grown from seed from selfed COIl/coil hemizvgous plants. Therefore, the F2 population was subjected to different treatments as indicated below, leaves from each individual collected separately and the plants further grown untill seed set. Individuals that did not form siliques were identified as having the coil/coil genotype. The coi 1 mutants used for the disease assays had been preselected based on root length of young seedlings germinated in vitro in the presence of 50 μM methyl jasmonate. All mutant lines listed above are derived from the Columbia (Col-O) ecotype. Growth and spore harvesting of the fungus A. brassicicola (MUCL 20297;Mycotheque Universite Catholique de Louvain, Louvain-Ia-Neuve, Belgiuni)was dorne as described previously (Broekaert et al., 1990).

Plant Growth Conditions, Chemical Application and Inoculation

nArabidopsis seed were sown on flower potting compost containing a macro-nutrient supplement (Asef. Didarn, The Netherlands) in petri-dishes. The seed were vernalized for 2 days at 4° C. following sowing. After 5 days of incubation in a growth chamber (20° C. day temperature, 18° C. night temperature, 12-hr photoperiod at a photon flux density of 100 μE m⁻²s⁻¹), seedlings were transferred to pots (5×4×4 cm) containing potting compost supplemented with macronutrients and grown under the same conditions as above. Irrigation was done with tap water via the trays carrying the pots. Leaves were wounded by crushing. with forceps with ribbed tips or by making incisions in the limb with a scalpel taking care to leave the midvein intact Paraquat (25 μM), rose bengal (20 mM), methyl jasmonate (45 μM in 0.1% (v/v) ethanol), 0.1% (v/v) ethanol, sodium salicylate (5 mM) and INA (1 mg/mL of a 25% active ingredient in a wettable powder, provided by Dr. H. Kessmann, Novartis, Basel, Switzerland) were applied at the concentrations indicated between brackets as 5 μL droplets on leaves (5 drops per leaf). The stock solution of methyl jasmonate was 45 mM in ethanol. Ethylene treatment was performed by placing pots in an air-tight translucent chamber in which gaseous ethylene was injected via a silicon rubber septum. The ethylene concentration in the chamber was verified by gas chromatography. Control plants for the ethylene experiment were placed in an identical chamber without ethylene. Inoculation with A. brassicicola was done by applying 5 μL drops of a spore-supension. (density of 5×10⁵ spores/mL in distilled water) on four lower rosette leaves (5 drops per leaf). Control plants were treated identically with water droplets. The plants with drops of spore suspension or water were placed randomly (different genotypes were treated simultaneously) in a propagator flat with a clear polystyrene lid and kept at high humidity for 2 days to stimulate infection by hyphal germlings. Thereafter, lids were taken off and the plants incubated further till harvesting of leaf material. The isolate of A. brassickola and inoculation conditions used here caused limited brown necrotic lesions under the drops of spore suspension within 48h of inoculation and these lesions failed to spread further.

RNA Blot Analysis

RNA was extracted by the phenol/LiCl method according to Eggermont et al. (1996) from tissues frozen in liquid nitrogen and stored at −80° C. RNA samples were loaded at 4 μg per lane on a formaldehyde-agarose gel and blotted onto a positively charged nylon membrane (Boehringer Mannheim, Mannheim, Germany) via capillary transfer with 20×SSC (Sambrook et al., 1989). To verify equal and transfer of RNA, the loading buffer was supplemented with 50 μg/mL ethidium bromide, allowing visualization of RNA in the gels and on the blots upon UV illumination. RNA was cross-linked on the blots by UV illumination of both sides for 5 minutes each. Blots were prehybridized, hybridized with digoxigenin-labeled antisense RNA probes and developed by immnunochemiluminescence as described before (Terras et al., 1995). Digoxigenin-labeled probes were made by run-off transcription using the Dig RNA labeling kit of Boehringer Mannheim. The PDF1.2 probe was synthesized using SP6 RNA polymerase and the EcoRI-linearized plasmid pZL1 (BRL life Technologies, Gaithersburg, Md., USA) containing the expressed sequence tag with Genbank accession number T04323. The probe for the tubulin β-1 chain gene was synthesized using T7 RNA polymerase and the EcoRI-linearized plasmid pBluescript II SK (Stratagene, La Jolla, Calif., USA) containing the expressed sequence tag with Genbank accession number Z26191. Both plasmids were obtained from the Arabidopsis Biological Resource Centre (Columbus, Ohio, USA). Samples analyzed with different probes were run on replicate gels which were developed separately.

Reverse Transcription PCR

Total RNA (1 μg in 100 mM sodium acetate/5 MM MgSO₄, pH 5.0) was treated with 6 units of RNase-free DNaseI (Boehringer Maunheim) at 37° C. for 5 minutes, whereafter the DNase was inactivated by heating at 95° C. for 5 minutes. The RNA was precipitated overnight in the presence of 0.2 M sodium acetate and 66% (v/v) ethanol, collected by centrifugation at 10000 xg for 10 minutes, washed twice with 70% (v/v) ethanol and finally dissolved in RNase-free water. The reverse transcription reactions were performed on 1 μg of DNase-treated total RNA with 10 units of avian myeloblastosis virus reverse transcriptase (Pharmacia, Uppsala, Sweden) for 60 minutes at 52° C. The reverse transcription reactions were performed with a homopolymeric deoxhymidine oligonucleotide(20-mer) and terminated by addition of Na-EDTA to a final concentrationof 5 mM. A fraction (one thirtieth) of the reverse transcription reaction solution was used as a template in a 50 μL PCR reaction performed with 2.5 units of Taq polymerase (Appligene, Pleasanton, Calif., USA) according to Sambrook et al. (1989). The PCR was run for 30 cycles with,an annealing temperature of 55° C., 65° C. and 65° C. for amplification with primer pairs specific for PDF1.1, PDF1.2 and ACTIN-1, respectively. Primers used for amplification of PDF1.1 were:

OWB260 (sense 5′-GAGAGAAAGCTTGTTGTGCGAGAGGCCAAGTGGG-3′); and

OWB259(antisense 5′-GAGAGAGGATCCTGCAAGATCCATGTCGTGCTTIC-3′), those for amplification of PDF1.2 were:

OWB240 (sense, 5′-AATGAGCTCTCATGGCTAAGTTTGCTTCC-3′); and

OWB241 (antisense, 5′-AATCCATGGAATACACACGATTTAGCACC-3′); and those for amplification of ACTIN-1 were:

OWB270 (sense, 5′-GGCGATGAAGCTCAATCCAAACG-3′); and

OWB271 (antisense. 5′-GGTCACGACCAGCAAGATCAAGACG-3′).

Purification of Plant Defensins from A. brassicicola-Infected Arabidopsis Leaves

Leaves of 5-week-old 4rabidopsis plants were inoculated with 5 μL drops of distilled H₂O (control) or a A. brassicicold spore suspension (5×10⁵ spores/mL in H₂O) and collected after 3 days of incubation in a moist propagator flat with a clear polysterene lid. Extracts were prepared from 20 g of either H₂O-treated or inoculated leaves and subjected to the purification procedure exactly as previously described in Terras et al. (1995). Protein determination, in vitro antifungal activity determination and SDS-PAGE analysis orrprecast PhastGel High Density gels (Pharmacia) were performed as previously described (Terras et al., 1995). Prior to SDS-PAGE analysis, protein samples were reduced with dithioervthritol and S-pyridylethylated as in Terfras et al. (1992). Immnunoblotting of proteins, separated on a 15% acryam SDS-PAGE gel was done as described in Terras et al. (1995).

ELISA Analysis

Proteins were isolated from frozen leaf material in extraction buffer (10 mM NaH₂PO₄, 15 mM Na₂HPO₄, 100 mM KCl, 1.5% (w/v) polyvinylpolypyrrolidone, pH 7). Protein concentrations were determined in the crude extracts according to Bradford (1976) using bovine serum albumin as a standard. After heat treatment (10 min. 80° C.) of the extract the heat-stable soluble protein fraction- was- analyzed in a competition ELISA.

ELISA Microtiterplates (Greiner Labortechnik) were coated with 100 ng/mL Rs-AFP2 in coating buffer (15 mM Na₂CO₃, 35, mM NaHCO₃, pH 9.6) for 2h at 37° C. The uncoated sites were blocked with 3% (w/v) cold fish skin gelatine (Sigma) in phosphate buffered saline (PBS) (2h, 37° C.). Affinity-purified primary antibodies were diluted 50-fold in 0.3% (w/v) gelatine in PBS, containing 0.05% (v/v) Tween20 and applied to the wells simultaneously with equal volumes (50 μL) of the samples diluted in the sample bufer. The plates were incubated for Ih at 37° C. After several washes with PBS containing 0.1% (v/v) Tween20, the plate wells were filled (100 μL per well) with secundary antibodies (goat anti-rabbit antibodies coupled to alkaline phosphatase, Sigma Immuno Chemicals) diluted 1000 fold in 0.3% (w/v) gelatine in PBS, containing 0.05% (v/v) Tween20. The plates were incubated for lh at 37° C. Alkaline phosphatase activity was measured after 30 to 60 min of incubation at 37° C. using the substrate 4-nitrophenyl phosphate (Janssen Chimica. Beerse, Belgium) at a concentration of 1 mg/mL in substrate buffer (20 mM Na₂CO₃. 35 mM NaHCO₃, 5 mM MgCl₂, pH 9.6) by absorbance measurement at 405 nmn. Protein samples were applied in 4-fold dilution series prepared in triplicate and the plant defensin concentration was measured relative to a two-fold dilution series of purified Rs-AFP1 (Terras et al., 1992) as a standard.

Affinity purification of anti-Rs-AFP 1 antiserum was done as follows. An antigen affinity column was prepared by mixing equal volumes of 20 mg/mL purified Rs-AFP1 in 100 mM 3-N-morpholinopropanesulfonic acid buffer (pH 7) with Affi-Gel 10 matrix-(Bio-Rad Laboratories. Hercules, Calif., USA) equilibrated in water. The slurry was incubated overnight at 4° C. with continuous gentle agitation. After blocking the unreacted sites of the matrix by addition of 0.025 volumes of 1 M ethanolamine (pH 8). the column was subsequently washed with 10 mM Tris (pH 7.5), 100 mM glycine (pH 2.5), 10 mM Tris (pH 8.8) and 100 mM triethylamine (pH 11.5), and equilibrated with 10 mM Tris (pH 7.5). The rabbit anti-Rs-AFP1 antiserum (Terras et al., 1995) was diluted two-fold in ImmunoPure Gentle Ag/Ab binding buffer (Pierce Chemical Company, Rockford, Ill., USA) and passed several times over the affinity column. After washing the column with 15 volumes of ImmunoPure Gentle Ag/Ab binding buffer, purified anti-Rs-AFP1 antibies were elditd with ImmunoPure Gentle Ag/Ab elutionbutder (Piercer Chernical Company). The elution of bound antibodies was monitored by measuring the absorbance at 280 nm. The eluted fraction was dialyzed overnight against PBS.

Amplification, Cloning and DNA Sequence Analysis of the Promoter Region of the Arabidopsis PDF1.2 Gene.

A strategy based on the inverse polymerase, chain reaction (for details of this method see Gasch et aL 1992) was used to amplify 5′regulatory sequences of the PDF1.2 gene. The DNA sequence of the expressed sequence tag (EST) with Genbank accession number T04323 that corresponds to the PDF1.2 cDNA was used to design primers for the amplification of 5′genomic flanking sequences from the Arabidopsis genome. Total genomic 30 DNA was isolated from 10 g fresh weight of leaves from approximately 8 week old plants of Arabidopsis ecotype Col-0 using the method of Dellaporta et al. (Dellaporta et al.). The DNA was further purified by resuspending in a CsCl solution at a final density of 1.55 g/ml and containing 0.75 mg/ml of ethidium bromide and centrifuging at 45.000 rpm. The banded DNA was then removed from the centrifuge tube by a syringe, the ethidium bromide removed by partitioning against isoarnyl alcohol and the DNA then precipitated by ethanol. The precipitated DNA was dissolved in water and 120 ng digested for 1 h at 37° C. with 10 units of the restriction enzyme Sphl in a 40 μl reaction. The EST T04323 was known to have an internal Sphl site (at bases 174 to 179) and therefore this enzyme was used to excise genomic fragments that would contain the first 178 bases of the cDNA, any intervening sequences and any 5′sequence upstream of the EST sequence to the first Sphl site in the genomic DNA. The reaction was heat inactivated at 65° C. for 10 min, centrifuged briefly and the DNA precipitated from the supernatant using ethanol. Approximately 30 ng of the digested DNA was then self-ligated overnight at 14° C. in a standard 50 μl reaction using 1 unit of T4 DNA ligase and a buffer provided by the enzyme supplier (Boehringer Mannheim). The ligation reaction was stopped by heating at 65° C. for 10 min, briefly centrifuged and the DNA precipitated from the supernatant using ethanol. A sample of 10 ng of the ligated DNA was then added as template to a 50 μl Polymerase Chain Reaction (PCR) containing 200 μM dNTPs and 1 μM of each of the primers OWB257 [5′-GAGAGAGGATCCAACTTCTGTGCTTCCACCATTGC-3′BamHIl site underlined] and OWB256 [5′-GAGAGAAACTTGAAGCCAAGTGGGACATGGTCAGG-3′, HindIII site underlined]. These primers correspond to sequences at positions 104-127 and 133-156 in the EST T04323 sequence, respectively, but are inverted and would only amplify if flanking sequences were aligned by self-ligation of the genomic fragment at the terminal Sphl sites. The PCR reaction contained 1 unit of Taq DNA polymerase (added when the reaction reached 95° C. in the first thermal cycle) and the reaction buffer recommended by the supplier (Appligene Inc.). The PCR reaction was subjected to the following thermal cycle regime. First, heating at 95° C. for 4 min during which the Taq polymerase enzyme was added, this followed by 30 cycles of 30 sec at 95° C., 2 min at 56° C. (annealing temperature) and 1 min at 72° C., and a final cycle of 30 sec at 95° C., 2 min at 56° C. and 10 min at 72° C. A sample equivalent to 0.05 μl of this reaction was added as template to a second PCR reaction that was identical except that no genomic DNA was added and that the nested primer OWB255 [5′-GAGAGAGGATCCAGCAGCAAAGAGAACAAGAGCAGCG-3′, BamHI site underlined] was added instead of primer OWB257. The primer OWB255 corresponds to positions 67-91 in the T04323 sequence. This reaction was subjected to the same thermal cycling regime except that the 56° C. step was increased to 58° C. Two DNA fragments approximately 1.3 kb and 0.8 kb in size were obtained. The larger band was isolated from the agarose gel, digested with HindIII and BamHI and ligated into pEMBL 18+predigested with HindIII and BamHI. This clone was termed pJMiPCR-lt. The insert was partially sequenced using dideoxynucleotide teminators and the M13 forward and M13 reverse primers. These partial sequences revealed that the insert contained sequences identical to those that would be predicted to be present from the EST T04323 sequence and partial sequences that flanked the Sph I restriction site used for the self-ligation were. identified. An oligonucleotide primer was then designed to the sequence adjacent to the Sphl site in the pJMiPCR-lt insert that was predicted to contain sequences located in the 5′promoter region of the PDF1.2 gene. This primer was termed OWB273 [5′-AAGAAAGCTTATGCATGCATCGCCGCATCGATATCCC-3′, HindIII site underlined]. This primer was used in a PCR reaction in combination with primer OWB276 [5′-GAGAGAAGCATTATTTTTATGTAAAATACACACGATTTAGCACC -3′, underlined] which contains sequence corresponding to positions 292-326 in the EST T04323 sequence. If the inverse PCR reaction had correctly agmplified 5′. upstream sequences of the PDF1.2 gene then these primers should amplify from genomic DNA all sequences of the PDF1.2 gene that are 5′to position 326 on the T04323 sequence and this region should extend as far as the Sphl site in the 5′upstream promoter region. Using 10 ng of intact genomic DNA of,Arabidopsis ecotype Col-0 as a template, a PCR reaction was carried out with these primers as described above using 58° C. as the annealing temperature. This PCR yielded a single fragment of approximately 1.6 kb in size as judged by gel electrophoresis. The band was excised from the gel and digested with HindIII and ligated into HindIII cut and dephosphorylated pEMBL 18+. This clone was termed pFAJ3085 and the insert was sequenced completely on both strands with overlap using fluorescently labelled dideoxynucleotide terminators and an Applied Biosystems 373A DNA Sequencer.

Construction of PDF1.2 Promoter-Reporter Constructs and Transformation of Arabidopsis and Tobacco

The promoter fragment and part of the coding sequence of the PDF1.2 gene (positions 1-1254 in pFAJ3085 insert) was amplified using primers OWB272 [5′-GAGAGAGGATCCGATGGAAGCAAACTTAGCCATG-3′, BamHI site underlined] and OWN273. This reaction used 1 ng of pFAJ3085 as template and was carried out as before using an annealing temperature of 56° C. The predicted fragment size was obtained and gel purified. This fragment was digested with both HindIII and BamHI and then ligated into the commercial binary vector plasmid pBI101.3 (Clontech Inc.). This vector contains a T-DNA region that can be transferred to plants using Agrobacterium tumefaciens as an intermediary. On the T-DNA is a selectable marker gene conferring kanamycin resistance when expressed in plant cells. In addition. there is a polylinker containing HindIll and BamnHI sites situated immediately 5′ of the coding region of the UidA cistron from Escherichia coli (encoding the enzyme β-glucuronidase or GUS), followed by the terminator of the nopaline synthase gene from Agobacterium tumefaciens (tNOS). Insetion of the HindIII/BamHI digested promotor of the PDF1.2 gene amplified by OWB272 and OWB273 (pPDF1.2 ) into this region leads to a gene fusion where one would predict that the initiation of translation at the predicted normal translation initiation codon of the PDF1.2 gene (positions 1233-1235 in pFAJ3085) would lead to production of a GUS fusion protein with the following N-terminal sequence: MAKFASIRIPGYGQSLM, where underlined amino acid residues are derived from the first seven N-terminal PDF1.2 codons. normal font indicates arino acid residues encoded on the linker region of the pBI101.3 vector and the final italicised M residue is the N-terminal residue of the GUS enzyme encoded by the UidA cistron. This vector containing the chimeric pPDF1.2-GUS-tNOS gene was termed pFAJ3086. The insert in pFAJ3086 was reamplified using OWB273 and a commercial primer [GUS Sequencing Primer, Clontech Inc. 5′-TCACGGGTTGGGGTTTCTAC-3′] and the terminal sequences of the PCR product directly sequenced to verify that the sequence of the PDF1.2 gene had been correctly incorporated.

Plasmid pFAJ3086 was then transferred to the Agrobacterium tumefaciens strain LBA4404 by triparental mating using an E. coli HB101 strain containing the vector pRK2013 to promote conjugation (Ditta et aL). The A. tumefaciens strain containing the pFAJ3086 vector was used to transform leaf explants of Nicotiana tabacum cv. Xanthi—nc by the leaf disc method (Horsch et al.) and to Arabidopsis thaliana Ecotype C24 using root explants (Valvekens et al.).

Disease Assays

The disease assay for Botrytis cinerea on Arabidopsis was performed as follows. Arabidopsis plants were grown on potting compost in a growth chamber (22° C., 14 hr photoperiod at a photon flux density of 80 μE m⁻²s⁻¹, 70% relative humidity). Three weeks after sowing, all expanded leaves were wounded by pricking (3 pricks per leaf) with a needle. The wound sites were covered with a 5 μl droplet of a suspension of Botrytis cinerea (10⁵ spores/ml) in half strength potato dextrose broth (Difco). The inoculated plants were-placed randomly in a propagator flat with a clear polystyrene lid and incubated as above except that the photon flux density was reduced to 50 μE m⁻²s⁻¹ After 2 days, the lids were taken off and the plants further incubated under the same conditions. Plants were considered dead when the stem including the shoot apex and the youngest leaves were completely decaved.

Susceptibility of Arabidopsis to Peronospora parasitica was tested as follows. Conidia of P. parasitica pathovar Wela were collected by gently shaking infected leaves of Arabidopsis ecotype Weiningen in water. The conidial suspension was adjusted to 10⁵ spores/ml and sprayed on four-week-old plants using a paint spray. The inoculated plants were placed randomly in a propagator flat with a clear polystyrene lid and incubated for 7 days at 20° C. with a photoperiod of 8 h at a photon flux density of 80 μE m⁻²s⁻¹. Disease progression was examined by staining the leaves for fungal structures using the-lactophenol/cotton blue staining method (Keogh et al.).

### EXAMPLE 1

A Plant Defensin with Antifungal Activity Accumulates in Pathogen-Stressed Arabidopsis Leaves

To investigate whether the pathogen-induced plant defensin in Arabidopsis leaves is biologically active, we attempted to purfy the protein by a method previously developed for the purification of Rs-AFP3 and Rs-AFP4 from fungus-infected radish leaves (Terras et al., 1995). In short, the purification method consists of passage of a crude leaf protein extract over an anion exchange column at pH 7.5, passage of the unbound proteins over a cation exchange column at pH 5.5, elution of the bound proteins at high ionic strength and, finally, separation of the eluted proteins over a reversed-phase chromatography column. Comparison of the reversed-phase chromatogram of proteins from leaves of non-inoculated Arabidopsis plants with that of proteins from leaves inoculated with A. brassicicola, led to the identification of a peak present only in the latter (peak 1 in FIG. 3A). SDS-PAGE analysis showed that the protein in this peak fraction runs as a single 5 kDa band comigrating with Rs-AFP1 (FIG. 3B and 3C). This protein was equally well recognized as Rs-AFPl in inmmunoblots prepared from SDS-PAGE gels and developed with anti-Rs-AFP1 antiserum (FIG. 3C). Furthermore, the protein in the peak 1 fraction was demonstrated to inhibit the growth in vitro of A. brassicicola (FIG. 3D) as well as that of Fusarium culmorum (results not shown). The growth inhibition was characterized by hyphal hyperbranching and tip swelling as observed for Rs-AFP1 (FIG. 3D) and related plant defensins from other Brassicaceae, including PDF1.1 (previously called At-AFP1) from Arabidopsis seed (Terras etal., 1993).

### EXAMPLE 2

Systemic Induction of Arabidopsis Plant Defensins upon Pathogen Stress

We have previously shown that plant defensin genes in radish leaves are systemically induced upon local infection with A. brassicicola (Teras et al., 1995). To investigate whether this is also the case in Arabidopsis, we analyzed the induction of plant defensins in Arabidopsis leaves infected with A. brassicicola and non-treated leaves of infected plants (systemic leaves), both by ELISA (using antigen affinity-purified anti-Rs-AFP1 antiserum) and by RNA gel blot analysis (using PDF1.2 as a probe for defensins). At the protein level, plant defensins were found to increase from amounts below the detection limit (0.05 μg/mg total soluble protein) at the sampling time just before inoculation to up to 10 and 3 μg/mg total soluble protein in the pathogen-treated leaves and non-treated, systemic leaves at 72h after inoculation, respectively (FIG. 4A). Steady state levels of defensin mRNA were raised following inoculation both in pathogen-treated and non-treated, systemic leaves and the mRNA amounts in both of these leaf samples reached a maximum at 48h after inoculation (FIG. 4B).

### EXAMPLE3

Induction of Arabidopsis Plant Defensins upon Chemical Treatments

Most thus far studied genes that are induced systemically upon pathogen stress can be induced at least partially by salicylic acid (SA) or 2,6-dichloroisonicotinic acid (INA), a synthetic compound mimicking the action of SA (Ward et al., 1991). In Arabidopsis, it has been shown that PR-1, PR-2 and PR-3 are strongly induced by both SA and INA treatment (Uknes et al). However, no induction of plant defensin gene expression could be observed upon SA or INA treatment, whether assessed by RNA blot analysis or by ELISA (FIG. 5). In contrast, application of ethylene or methyl jasmonate significantly increased the level of plant defensin gene expression. No increase in plant defensin gene expression was detected in the appropriate control treatments i.e. incubation in air (control for ethylene) or application of 0.1% (v/v) ethanol (solvent control for methyl jasmonate). The expression of PR-protein genes (PR-1, PR-2, PR-3) in Arabidopsis has previously been shown not to respond to ethylene treatment (Lawton et al. 1994). Taken together, these data indicate that plant defensin genes and PR-protein genes are induced by different groups of chemicals.

The chemicals paraquat and rose bengal were also tested for their effect on expression of plant defensin genes. Treatment of plants with paraquat or rose bengal is known to result in the formation of the reactive oxygen species superoxideanion and singlet oxyen, respectively (Bowler et al., 1992; Green and Fluhr, 1995), and hence to cause oxidative stress. Plant defensins protein and MRNA-were strongly induced by either paraquat or rose bengal (FIG. 5).

Wounding of Arabidopsis leaves did not result in accumulation of detectable;levels of plant defensin mRNA when assessed 48h after treatment (FIG. 5). Because a number of wound-induced genes are known to be switched on transiently at relatively short time periods after treatment (Berger et al., 1995; Warner et al., 1993) we also verified plant defensin gene expression, both by RNA blot analysis and ELUSA, at 3, 6, 9, 12, 24 and 48h after wounding. However, expression of plant defensin genes could not be detected atany of the analyzed time points, irrespective of whether wounding was applied by making incisions in the leaves with a scalpel or by crushing the leaves with forceps (results not shown).

### EXAMPLE 4

Systemic Induction of Plant Defensins in Arabidopsis Follows a Jasmonate and/or Ethylene-Dependent Pathway

Because SA and INA were unable to induce the synthesis of plant defensins, we further investigated the role of SA in the induction of plant defensins following fungal infection. Therefore, we measured plant defensin gene expression in Arabidopsis plants known to be genetically modified in the SA-signalling pathway. The npr1 mutant displays a blocked SA-signalling pathway as if is unable to express PR-protein genes upon SA treatment or pathogen infection (Cao et al., 1994). In contrast, the cpr1 mutant has constitutively elevated SA and PR-protein levels even in the absence of any stress signal.

Plants of different Arabidopsis lines affected in the SA-signalling pathway were either mock-inoculated with water or inoculated with an A. brassicicola spore suspension and treated and non-treated (systemic) leaves were harvested after 72h. The expression of plant defensin genes was measured both by RNA blot analysis and ELISA. When wild-type (Col-0) plants were inoculated with A. brassicicola, expression of plant defensin genes was induced in both pathogen-treated leaves and non-treated, systemic leaves when compared to that of the corresponding leaves of mock-inoculated plants (FIG. 6). In both the npr1 mutant and the cpr1 mutant plant defensin gene expression was similarly induced upon challenge with A. brassicicola and no constitutive expression was observed in a mock-inoclated cpr1 plants.

### EXAMPLE 5

The Pathway for Induction of Plant Defensins Requires Components of the Ethylene and Jasmonate Response Pathways

As shown above, plant defensins accumulate in leaves of Arabidopsis plants after-application of exogenous ethylene or methyl jasmonate. To clarify the role of these two plant hormones in the induction of plant defensins, we investigated expression of plant defensin genes in the ethylene-insensitive mutants ein2 and etr 1-3 (Guzman and Ecker, 1990; Chang et al., 1993) as well as in the coronatine—and methyl jasmonate-insensitive mutant coil (Feys etal., 1994).

The induction of plant defensins was markedly affected upon fungal infection of the ethylene-insensitive or jasmonate-insensitive mutants. The induction of plant defensins appeared to be almost totally blocked in ein2 and coil plants. The levels of plant defensins in pathogen-treated and non-treated, systemic leaves of ein2 and coil plants after inoculation with A. brassicicola were at least 30-fold lower than the levels in the corresponding leaf samples of similarly pathogen-treated wild-type plants. The block in pathogen-induced expression of plant defensin genes in ein2 and coil plants probably occurred at the transcriptional level since no plant defensin mRNA was detected in leaves of pathogen-treated plants. In etr1-3 plants, expression of plant defensin genes was enhanced in the pathogen-treated leaves to levels similar to those found in pathogen-treated leaves of wild-type plants. However, in non-treated, systemic leaves of etr1-3 plants, accumulation-of plant defensins was increased to a level that was three-fold lower than in the systemic leaves of wild-type plants. This reduction was consistent throughout three independent experiments but the extent of reduction was variable.

### EXAMPLE 6

Plant Defensins are Constitutively Induced in the Lesion Mimic Mutant acd2

The Arabidopsis acd2 mutant spontaneously develops lesions similar to those developed by wild-type plants undergoing a hypersensitive response upon challenge with avirulent bacterial pathogens (Greenberg et al., 1994). Sifice this mnutant has previously been shown to accumulate high levels of PRiprotein g-eie transcripts in both asymptomatic and necrotic leaves, (Greenberg et al., 1994), it was considered worthwhile to assess plant defensingene expression in acd2 plants. Healthy asymptomatic upper rosette leaves and lower rosette leaves displaying necrotic lesions were harvested separately from 5-week-old acd2 plants, as well as healthy upper and lower rosette leaves from wild-type (Col-0) plants grown under the same conditions. As measured by ELISA, acd2 plants accumulated very high levels of plant defensins, estimated to constitute about 5% and 10% of total soluble proteins in asymptomatic leaves and leaves with necrotic lesions, respectively (FIG. 8B). RNA blot analysis showed that transcript levels of plant defensins in necrotic as well as in asymptomatic leaves of acd2 were strongly elevated compared to those in wild-type plants (FIG. 8A).

### EXAMPLE 7

Ethylene and Jasmonic Acid Activate the PDF1.2 Gene Via Parallel Signalling Paths.

The observation that pathogen-induced expression of PDF1.2 is blocked both by a mutant affected in the ethylene response (ein2) as well as by a mutant affected in the jasmonate response (coil) implies that ethylene and jasmonate somehow interact with each other to effect expression of this gene. Conceptually, three different models for the interaction between ethylene and jasmonate can be conceived (FIG. 10).

A first model implies that pathogen recognition leads to increased ethylene production which in turn would result in stimulated jasmonate production and subsequent PDF1.2 activation A second model would be identical to the first. except that the hierarchy between the ethylene and jasmonate signals would be reversed. A third model finally, supposes that ethylene and jasmonate do not act in a sequential manner but rather via parallel pathways which both need to be activated for induction of the PDF1.2 gene upon pathogen recogniuon.

It was previously shown that inoculation of Arabidopsis wild type plants (Col-O) with Alternaria brassicicola results in an increase in the levels ofjasmonic acid in the leaves (Penninckx et al. 1996). The first model predicts that such a rise in jasmonate level upon pathogen infection would not occur in the ethylene-insensitive mutant ein2, whereas according to models 2 and 3, the ein2 mutation would not affect pathogen-stimulated jasmonate production. To test these divergent predictions, ein2 and wild type plants (Col-O) were infected with the pathogen A. brassicicola whereafter jasmonate levels were monitored at different time points. As shown in FIG. 11, jasmonate levels in fungus-inoculated wild type plants started to rise as from 24 h after inoculation and more dramatically as from 48 h after inoculation to reach a peak level at about 72 h postinoculation. In the coil mutant, pathogen-stimulated production was clearly not abolished but even more pronounced relative to the wild type plants. Hence, these results are in conflict with the predictions made based on model 1.

To further discriminate between models 2 and 3, ethylene production by wild type plants and coil mutants was measured in response to inoculation with A. brassicicola. Model 2 predicts that the coil mutant would be blocked in its ability to stimulate ethylene production upon pathogen attack, while model 3 implies that the ethylene response in the coil mutants would not be reduced versus that of wild type plants. Inoculation of wild type plants with A. brassicicola resulted in ethylene production levels that were about twice those in mock-inoculated plants, with a peak level reached at about 60 h after inoculation (FIG. 12). A similar two-fold increase in ethylene production levels was also observed in the coil mutant plants treated with A. brassicicola (FIG. 12). The ethylene production levels in both inoculated and mock-inoculated coil mutants were on average about two-fold higher relative to those in wild type plants. As the pathogen-stimulated ethylene production was clearly not abolished in the coil mutants, it was concluded that model 2 is not valid. Model 3, proposing parallel ethylene and jasmonate signalling paths, is hence the only model that is in agreement with all observations.

An alternative way to verify the validity of model 1 is to treat wild type plants and ein2 mutants with methyl jasmonate and subsequently measure plant defensin levels by ELISA two days after the treatment. Treatment of wild type plants with 50 μM methyl jasmonate resulted in a plant defensin level that was at least 100-fold higher relative to that in solvent-treated wild type plants. In contrast, the plant defensin content of ein2 mutants treated with methyl jasmonate was not elevated relative to that in solvent-treated plants (FIG. 13). This observation is in conflict with model 1 which predicts that jasmonate-induced PDF1.2 accumulation would not be affected by the ein2 mutation.

The fact that treatment with methyl jasmonate alone is-able to activate the PDF1.2 gene is not necessarily in conflict with model 3 as it is well established that treatment of plants by external application of methyl jasmonate results in enhanced production of ethylene (Czapski and Saniewski, 1992). Treatment of non-sterile soil-grown plants with ethylene alone results in enhanced plant defensin accumulation (see Example 3), which is also in apparent disagreement with model 3. However, when sterile plants grown on an agar medium were treated with ethylene alone (25 ppm), no induced PDF1.2 accumulation was observed (results not shown). Treatment of sterile plants with methyl jasmonate still induced PDF1.2 accumulation, most probably because of simultaneous stimulation of ethylene production. The observation that ethylene stimulates PDF1.2 accumulation in non-sterile plants but not in sterile plants might be explained by assuring-that contact of plants with micro-organisms in the soil alters their responsiveness to ethylene treatment.

It is to be noted that whilst model 3 is considered to be the most likely model, it may be shown with further experimentation that one of the other models is more likely to be the model which is best describes the interaction between ethylene and jasmonate.

### EXAMPLE 8

Development of a Scorable Marker Gene for the Induction of the Jasmonate and/or Ethylene-Dependent Defence Pathways in Arabjdopsis and Tobacco.

The Arabidopsis PDF1.2 gene promoter was cloned via an inverse PCR strategy using primers based on the PDF1.2 cDNA sequence (genbank accession number T04323). This procedure resulted in the cloning of a 1616 bp genomic DNA fragment whose sequence is shown in FIG. 14. The sequence of this genomic fragment at positions 1202-1296 and 1388-1616 matched exactly the sequence of the PDF1.2 cDNA from positions 1-326. The interuption in the match-of the suence at positions 1297-1387 relative to the cDNA sequence is presumed to represent a 91 bp intron which is situated within the coding sequence for the PDF1.2 signal peptide. The sequences surrounding the predicted intron splice sites were consistent with consensus sequences observed in other plant genes. Importantly, the genomic fragment contained 1201 bp 5′of the cDNA sequence and 1232 bp upstream of the predicted translational start of the PDF1.2 gene product. Experiments were then designed to test whether the DNA sequence 5′of the translational start codon of the PDF1.2 gene might contain a promoter with regulatory elements that determine pathogen-induced local and systemic expression of this gene as well as induction by jasmonates and other chemical stimulants that will induce accumulation of the PDF1.2 gene product. To this end, the first 1254 bp of the genomic fragment encompassing the putative promoter elements together with the 5′untranslated leader and a part of the region encoding the first seven PDF1.2 codons was linked as a translational fusion to the coding region of the Escherichia coli UidA gene (encoding β-glucuronidase or GUS) which in turn was hooked up to the Agrobacterium tumefaciens nopaline synthase gene terminator (tNOS) . The resulting pPDF 1.2-GUS-tNOS expression cassette was transferred within a plant transformation vector into Arabidopsis thaliana ecotype C24 by Agrobacterium tumefaciens—based root transformation.

The ability of the pPDF1.2-GUS-tNOS transgene to respond to jasmonic acid treatment was first assessed on the progeny of six independent T0 transgenic Arabidopsis lines. Seeds of each of these lines were germinated aseptically, either on Murashige and Skoog agar medium containing 1% sucrose and 50 μM kanamycin or the same medium supplemented with 10 μM jasmonic acid. Seedlings were harvested 10 days after mination and their β-glucuronidase activity measured fluorometrically using 4-methylumbelliferyl glucuronide as a substrate according to the procedure of Jefferson (1987). The β-glucuronidase activity was normalized to the total protein content in each sample, as determined by the method of Bradford (1976) using bovine serum albumin as a starrdard.

As shown in Table 1, all lines tested showed a marked increase (ranging from 9 to 25 fold) of the β-glucuronidase activity in the jasmonate treated seedlings versus the untreated seedlings. Line 19, which showed the strongest relative jasmonate-induced increase of β-glucuronidase activity was further bred by self-pollination to obtain T3 generation seed that was homozygous for the transgene. This line was used in all subsequent analyses.

TABLE 1
β-glucuronidase activity in 10-day-old seedlings of six independent
Arabidopsis lines carrying the pPDF1.2-GUS-tNOS transgene after
germination in the absence or the presence of 10 μM jasmonic acid.

β-glucuronidase activity*

| Transgenic line | control | JA-treated | Fold induction |
|---|---|---|---|
| 3 | 115 | 2895 | 25.2 |
| 4 | 113 | 978 | 8.7 |
| 5 | 296 | 2676 | 9.0 |
| 6 | 149 | 2166 | 14.5 |
| 7 | 21 | 203 | 9.7 |
| 19 | 8 | 202 | 25.2 |
| *expressed as pmoles 4-methylumbelliferone released from 4-methylumbelliferyl glucuronide at 37° C. | | | |
| # per min and per mg soluble protein | | | |

The PDF1.2 gene was shown by RNA gel blot analyses and ELISA assays to be systemically induced upon inoculation of Arabidopsis leaves with the fungal pathogen Alternaria brassicicola. The expression of the pPDF1.2-GUS-tNOS reporter gene in transgenic four-week-old Arabidopsis plants in response to inoculation by the fungal pathogens Alternaria brassicicola and Botrytis cinerea, was assessed by β-glucuronidase activity measurements both in inoculated and non-inoculated systemic leaves 48 h following treatment of the transgenic plants. A. brassicicola was inoculated by applying 5 μl drops of a conidial spore suspension (5×10⁵ spores/ml in water) on the lower rosette leaves (4 drops per leaf). B. cinerea was inoculated as 5 μl drops of a 5×10⁵ spores/ml conidial spore suspension in half strength potato dextrose broth (Difco) covering a needle prick wound applied on the lower rosette leaves (1 drop per wound, 4 wounds per leaf). As shown in FIG. 15, β-glucuronidase activity was strongly increased following inoculation with either A. brassicicola or B. cinerea both in the inoculated leaves and the non-inoculated, systemic leaves, relative to the mock-inoculated controls. This indicates that the pPDF1.2-GUS-tNOS reporter gene is a suitable marker for systemic pathogen-induced gene expression in Arabidopsis.

Four-week-old pPDF1.2-GUS-tNOS transgenic Arabidopsis plants were treated with various chemicals to investigate their ability to induce expression of the reporter gene. The chemicals used in these tests were jasmonic acid (JA, 500 μM in 0.1% ethanol), methyl jasmonic acid (MeJA, 50 μM in 0.1% ethanol), salicylic acid (SA, 5 mM in H₂O), 2,6-dichloroisonicotinic acid (INA, 1 mg/ml in H₂O), 1,2,3-benzothiadiazole-7-carbothioic acid S-methylester (BTH, 300 μM in H₂O), paraquat (PQ, 25 μM in H₂O). The chernical solutions were applied on the fully expanded leaves of the plants as 5 μl drops deposited on the upper leaf surface (5 drops per leaf). A separate set of plants was also treated by exposure to ethylene (25 ppm) in an air-tight chamber. Following treatment for 48 hours with either the chemicals or the appropriate controls, leaves from treated plants were harvested and β-glucuronidase activity measured.

As shown in FIG. 16, treatment with either JA, MeJA, or paraquat induced the pPDF 1.2-GUS-tNOS reporter gene, whereas applications of either SA, INA, BTH or ethylene were without effect. These findings corroborate the expression studies based on RNA gel blot analyses and ELISA assays of the PDF1.2 gene in response to chemical treatments, except for ethylene treatment. Although ethylene treatment was found to increase PDF 1.2 levels in non-sterile soil-grown plants (see Example 3), this gaseous hormone did not boost PDF 1.2 accumulation in sterile plants (see Example 7). Hence, non-sterile culture conditions appear to increase the responsiveness of Arabidopsis plants to ethylene. This increased responsiveness may be sufficient to activate the endogenous PDF1.2 gene but not the pPDF1.2-GUS-tNOS chimeric reporter gene.

To verify whether SA, INA and BTH were applied under conditions that would induce SA-dependent pathogen-induced genes, the above tests were also performed on transgenic Arabidopsis plants carrying a chimeric transgene consisting of the β-glucuronidase coding region under the control of the promoter of the Arabidopsis β-1.3-glucanase PR-2 type gene, named Bg12 (Bowling et al., 1994, Plant Cell 6, 1485-1857). This chimeric gene is hereunder abbreviated as pBg12-GUS-tNOS. As shown in FIG. 3, treatment of transgenic pBg12-GUS-tNOS Arabidopsis plants with SA and its functional analogs INA and BTH did result in markedly increased β-glucuronidase activity, whereas neither JA, MeJA, ethylene nor paraquat had any effect. It has previously been shown that SA, INA and BTH induce SA-dependent pathogen-inducible genes in Arabidopsis such as PR-1, PR-2 and PR-5 (Uknes et al., 1992).

Taken together, these data indicate that thhp PDF1.2-GUS-tNOS reporteregene responds systemically to pathogen infection without the intervenence of SA, indicating that it is an appropriate scorable marker for SA-independent pathogen-induced gene activation and possibly for SA-independent induced resistance.

In order to assess whether the pPDF1.2-GUS-tNOS promoter could be used as a marker for SA-independent pathogen-induced gene expression in a plant other than Arabidopsis, the pPDF1.2-GUS-tNOS gene was also introduced into the tobacco cultivar Xanthi-nc by Agrobacterium-mediated leaf disc transformation. T2 generation plants were selected that were homozygous for the transgene. Systemic pathogen-induced expression-in a transgenic tobacco line was assessed on 8-week-old plants after inoculation with tobacco mosaic virus of the leafjust below the youngest fully expanded leaf. Tobacco cultivar Xanthi-nc is resistant to tobacco mosaic virus and reacts to the virus by producing a hypersensitive response, thus preventing the virus from spreading beyond the lesions. The youngest fully expanded leaf and the leaf above the youngest fully expanded leaf were harvested separately at 2, 4 and 6 days after inoculation and the β-glucuronidase activity was measured. Tobacco mosaic virus inoculun was prepared by grounding a leaf of tobacco cultivar Hicks preinfected with tobacco mosaic virus in 50 mM sodiun phosphate buffer (pH7) at 1 g tissue per 10 ml buffer. The suspension was diluted 10-fold in buffer and applied on tobacco leaves by rubbing with carborundum powder. Control plants were mock inoculated with buffer and powder only. As shown in FIG. 17, β-glucuronidase activity was markedly increased in the inoculated leaves as from two days after inoculation and in the systemic leaves as from four days after inoculation. In addition, the inducibility of the reporter gene was assessed by treating leaves with salicylic acid (SA, S mM in H₂O), methyl jasmonic acid (MeJA, 50 μM in 0.1% ethanol) paraquat (PQ, 25 μgM in H₂O) and by wounding. The chemicals were applied as four droplets of 1001 μl on the fully expanded leaf of 8-week-old plants. Wounding was tested by crushing leaves at 2 cm intervals with forceps with serrated ends. The β-giucturomidase content in the treated leaves and appropriate controls was measured 48 h after treatment.

As shown in FIG. 18, both methyl jasmonate and paraquat caused a more than 35-fold increase in β-glucuronidase activity. In contrast, wounding and salicylic acid treatment led only to a 2-fold increase in activity. Verification of the activation of the tobacco PR-1 gene by RNA gel blot analysis in the tobacco plants treated in the same way indicated that, like in the case of Arabidopsis, this gene was induced only by salicylic acid but not by methyl jasmonate, paraquat or wounding (not shown).

These results indicate that the PDF1.2 promoter is activated in a SA-independent way in tobacco, a plant from the family Solanaceae, while it can be induced systemically throughout the plant upon pathogen challenge. Hence, the pPDF1.2-GUS-tNOS reporter gene can be used in different plants for the purpose of screening compounds, microorganisms or physical treatments that induce the SA-independent defence pathway.

### EXAMPLE 9

Synergistic induction of the plant defensin promoter by ethylene and jasmonate.

Our finding that jasmonate and ethylene act via parallel paths to activate the PDF1.2 gene (see Example 7) suggests that combinations ofjasmonate and ethylene may have a synergistic effect on the activation of this gene. To test this hypothesis, ethylene and methyl jasmonate were combined at doses whereby each of both compounds alone could not activate the pPDF 1.2-GUS-tNOS gene in the transgenic Arabidopsis reporter plants. As shown in FIG. 19, treatment of Arabidopsis leaves with drops of 0.5 μM methyl jasmonate in an atmosphere containing 25 ppm ethylene resulted in a very high β-glucuronidase activity, whereas each single treatment did not cause increased P-glucuronidase levels. A significant but lower level of synergy was detected upon combining ethephon (a compound that generates ethylene when sprayed on plants) at 333 μM and methyl jasmonate at 0.5 μM (FIG. 19). In this test, ethephon was applied at a rate that was 10-fold lower than the rate normally used to activate senescence processes in plants.

These findings suggest that treatment of plants with a mixture of compounds that separately activate the ethylene response and the jasmonate response paths may result in a more effective stimulation of natural resistance against particular types of pathogens.

### EXAMPLE 10

Evidence that the Jasmonate and/or Ethylene-Dependent Defence Pathways Plays an Important Role in Defence OfArabidopsis Against Some Pathogens.

To test the disease susceptibility of Arabidopsis mutants affected in the jasmonate and/or ethylene dependent defence pathways, a disease bioassay was developted for the Ascomycetous necrotrophic fingai pathogen Botrytis cinerea (teleomorph=Botryotinia fuckeliana). This assay consisted ofapplying drops of a Botrytis cinerea spore suspension over needle prick wounds, whereafter succumbing of inculated plants was monitored. FIG. 20 shows the number of deceased plants in tests set up with series of wild type Arabidopsis plants (Col-0), ethylene insensitive mutants (ein2), and jasmonate inrsensitive mutants (coil). On average, 41% of all inoculated ein2 mutants and 86% of all inoculated coil mutants succumbed after 16 days, versus only 1% of the wild type plants (FIG. 20). Decay of the ein2 and coil plants was followed by abundant formation of conidia Preliminary tests indicated that npr1, a mutant affected in the salicylate-dependent defence response, was as resistant to Botrytis cinerea infection as the Col-O wild type plants. These results point to the importance of thejasmonate and/or ethylene-dependent defence response pathways for establishing resistance of Arabidopsis plants against the grey mould fungus Botrytis cinerea.

Wild type plants (Col-0) and the mutants npr1, ein2, and coil were also subjected to a disease bioassay performed with the Oomycetous biotrophic fugal pathogen Peronospora parasitica pathovar Wela. The infection by this fungus was assessed by detection of fungal structures in inoculated leaves using the lactophenol/trypan blue staining method. These tests showed that Col-O, ein2 and coil did not support any detectable growth of the pathogen whereas the npr1 mutant was heavily infected by fungal hyphae that abundantly formed oospores (FIG. 21).

Taken together these data indicate that the jasmonate and/or ethylene-dependent defence pathways described here is a key determinant in successful defence against infection attempts by Botrytis cinerea but not by Peronospora parasitica. and vice versa for the salicylate-dependent defence pathway. Hence. at least two distinct defence pathways that are effective against separate classes of pathogens appear to operate in plants. Importantly, this finding implicates that a broader spectrum of induced resistance will be reached upon treatment of plants with mixtures of compounds that induce both the jasmonate and/or ethylene-dependent defence pathways and the salicylate-dependent defence pathway.

If the jasmonate and/or ethylene-dependent pathogen-inducible defence response plays a pivotal role in host defence against some pathogens, as deduced from the enhanced disease susceptibility phenotype observed for ethylene insensitive and jasmonate insensitive Arabidopsis mutants, then it can be expected that pretreatment of plants with compounds activating this response would result in reduced susceptibility to certain pathogens.

Against at least B. cinerea we have shown that at least Arabidopsis plants can mount resistance by activating jasmonate and/or ethylene-dependent genes and not salicylate-dependent genes, which may explain the lack of protection conferred by 1,2,3-benzothiadiazole-7-carbothioic acid S-methylester against this pathogen (Lawton et al.).

Nearly all crop plants are susceptible to a range of pathogens rather than to one or two pathogenic microorganisms. It is therefore important that chemicals used for disease control confer resistance to a spectrum of pathogens that is as broad as possible. We have described here previously unidentified defence pathways, called the jasmonate and/or ethylene-dependent defence pathways, that not only differs genetically from the well-studied salicylate-dependent defence pathway but also contributes to the resistance against particular pathogens that are otherwise not controlled via activation of the salicylate-dependent defence pathway. It follows from these observations that a broader disease control spectrum can be obtained by treating plants with cocktails and compounds that activate both the salicylate-dependent defence pathway and the jasmonate and/or ethylene dependent defence pathways.

Other modifications of the present invention will be apparent to those skilled in the art without departing from the scope of the invention. For example, the screen of the present invention may also be used for screening for chemical. biological microbial or physical treatments of plants that result in salicylate-independent increased resistance to disease or pests.

REFERENCES

Beffa, R., Szell, M., Meuwly, P., Pay, A., Vögeli-Lange, R., Mètraux, J.-P., Neuhaus, G., Meins, F., and Nagy, F. (1995). Cholera toxin elevates pathogen resistance and induces pathogenesis-related gene expression in tobacco. EMBOJ 14, 5753-5761.

Bent, A. F., Innes, R. W., Ecker, J. R., and Staskawicz, B. J. (1992). Disease development in ethylene-insensitive Arabidopsis thaliana infected with virulent and avirulent Pseudomonas and Xanthomonas pathogens. Molec. Plant-microbe Interact. 5, 372-378.

Berger, S., Bell, E., Sadka, A., and Mullet, J. E. (1995). Arabidopsis thaliana Atvsp is homologous to soybean VspA and VspB, genes encoding vegetative storage protein acid phosphatases, and is regulated similarily-by methyl jasmonate, wounding, sugars, light and phosphate. Plant Mol. Biol. 27, 933-942.

Bleecker, A. B., Estelle, M. A., Somerville, C., and Kende, H. (1988). Insensitivity to ethylene conferred by a dominant mutation in Arabidopsis thaliana. Science 241, 1086-1089.

Bowler, C., Van Montagu, M., and Inzè, D. (1992). Superoxide dismutase and stress tolerance. Annu. Rev. Plant Physiol. Plant Mol. Biol. 43,-83-116.

Bowling, S. A., Guo, A., Cao, H., Gordon, A. S., Klessig, D. F., and Dong, X. (1994) A mutation in Arabidopsis that leads to constitutive expression of systemic acquired resistance. Plant Cell 6, 1845-1857.

Bradford, M. M. (1976). A rapid and sensitive method for the quantification of microgram quantities of protein utilizing the principle of protein-dye binding. Anal. Biochem. 72, 248-254.

Brederode, F. T., Linthorst, H. J. M., and Bol, J. F. (1991). Differential induction of acquired resistance and PR gene expression in tobacco by virus infection, ethephon treatment, UV light and wounding. Plant Mol. Biol. 17, 1117-1125.

Broekaert, W. F., Terras, F. R. G., Cammue, B. P. A., and Vanderleyden, J. (1990). An automated quantitative assay for fungal growth. FEMS Microbiol. Lett. 69, 55-60.

Broekaert, W. F., Terras, F. R. G., Cammue, B. P. A., and Osborn, R. (1995). Plant defensins : Novel antimicrobial peptides as components of the host defence system. Plant Physiol. 108, 1353-1358.

Cao, H., Bowling, S. A., Gordon, A. S., and Dong, X. (1994). Characterization of an Arabidopsis mutant that is nonresponsive to inducers of systemic acquired resistance. Plant Cell 6, 1583-1592.

Czapski and Saniewski (1992) J. Plant. Physiol. 139, 265-268.

Chang, C., Kwok, S. F., Bleecker, A. B., and Meyerowitz, E. M. (1993). Arabidopsis ethylene-response gene etrl: Similarity of product to two-component regulators. Science 262, 539-544.

Chen, Q. G., and Bleecker, A. B. (1995). Analysis of ethylene signal-transduction kinetics associated with seedling-growih responses and chitinase induction in wild-type and mutant Arabidopsis. Plant Physiol. 108, 597-607.

Cohen, Y., Gisi, U., and Nickerman, T. (1993). Local and systemic protection against Phytophthora infestans induced in potato and tomato plants by jasmonic acid and jasmonic-methyl-ester. Phytopathol. 83, 1054-1062.

Delaney, T. P., Uknes, S., Vernooij, B., Friedrich, L., Weymann, K., Negrotto, D., Gaffney, T., Gut-Rella, M., Kessmann, H., Ward, E., and Ryals, J. (1994). A central role of salicylic acid in plant disease resistance. Science 266, 1247-1250.

Dellaporta, S. L. J., Wood, J., Hicks, J. B. (1983) A plant DNA minipreparation: Version II. Plant Molecular Biology Reporter 1: 19-21

Dempsey, D. M. A., Wobbe, K. K., and Klessig, D. F. (1993). Resistance and susceptible responses of Arabidopsis thaliana to Turnip crinkle virus. Phytopathol. 83, 1021-1029.

Ditta, G., Stanfield, S., Corbin, D., Helinski, D.R. (1980) Broad host range DNA cloning system for Gram-negative bacteria: construction of a gene bank of Rhizobium meliloti. Proc. Natl,Acad. Sci. USA. 12, 7347-7351.

Doares, S. H., Narvaez-Vasquez, J., Conconi, A., and Ryan, C.A. (1995). Salicylic acid inhibits synthesis of proteinase inhibitors in tomato leaves induced by systemin and jasmonic acid. Plant Physiol. 108, 1741-1746.

Ecker, J. R., and Davis, R. W. (1987). Plant defence genes are regulated by ethylene. Proc. Natl. Acad. Sci. USA 84, 5202-5206.

Ecker, J. R. (1995). The ethylene signal transduction pathway in plants. Science -268, 667-675.

Eggermont K., Goderis I. J., Broekaert W. F. (1996) High-throughput RNA extraction from plant samples based on homogenisation by reciprocal shaking in the presence of a mixture of sand and glass beads. Plant Mol. Biol. Rep. 14, 273-279.

Epple, P., Apel, K., and Bohlmann, H. (1995). An Arabidopsis thalidna thionin gene is inducible via a signal transduction pathway different from that for pathogenesis-related proteins. Plant Physiol. 109, 813-820.

Feys, B. J. F., Benedetti, C. E., Penfold, C. N., and Turner, J. G. (1994). Arabidopsis mutants selected for resistance to the phytotoxin coronatine are male sterile, insensitive to methyl jasmonate, and resistant to a bacterial pathogen. Plant Cell 6, 751-759.

Friedrich et aL (1996) Plant J. 10, 61-70.

Gasch, A., Aoyana, T., Foster, R. and Chua, N-H. (1992). Gene Isolation with the polymerase Chain Reaction In Methods in Arabidopsis Research C. Koncz, N-H Chua, J. Schell (eds.) World Scientific Publishing Co. Ltd. Singapore pp 342-356.

Gorlach et al. (1996) Plant Cell, 8, 626-643.

Green,R., and Fluhr, R. (1995). UV-B-induced PR-l accumulation is mediated by active oxygen species. Plant Cell 7, 203-212.

Greenberg, J. T., Guo, A., Klessig, D. F., and Ausubel, F. M. (1994). Programmed cell death in plants a pathogen triggered response activated coordinately with multiple defence functions. Cell 77, 551-563.

Guzman, P., and Ecker, J. R. (1990). Exploiting the triple response of Arabidopsis to identify ethylene-related mutants. Plant Cell 2, 513-523.

Heitz, T., Fritig, B., and Legrand, M. (1994). Local and sytemnic accumulation of pathogenesis-related proteins in tobacco plantsirifected with tobacco mosaic virus.iMolec. Plant-Microbe Interact. 7, 776-779.

Horsch, R. B., Fry, J. E., Hoffmann, N. L., Eichholtz, D., Rogers, S. G. and Fraley, R. T. (1985) A simple and general method for transferring genes into plants, Science 227, 1229-1231.

Jefferson (1987) Plant Mol. Biol. Rep. 5, 387-405.

Keogh et aL (1980), Trans. Br. Mycol. Soc. 74, 329-333.

Kessmann et al. (1994) Annu. Rev. Phytopathol. 32, 439-459.

Kogel et al. (1995). Eur. J. Plant Pathol. 101, 319-332.

Lawton, K., Weymann, K., Friedrich, L., Vernooij, B., Uknes, S., and Ryals, J. (1995). Systemic acquired resistance in Arabidopsis requires salicylic acid but not ethylene. Mol. Plant-Microbe Interact. 8,863-870.

Lawton, K. A., Potter, S. L., Uknes, S., and Ryals, J. (1994). Acquired resistance signal transduction in Arabidopsis is ethylene independent.- Plant Cell 6, 581-588.

Lawton et aL (1996) Plant J. 10, 71-82.

Mauch, F., Hadwiger, L. A., and Boller, T. (1984). Ethylene: Symptom, not signal for the induction of chitinase and β-1,3-glucanase in pea pods by pathogens and elicitor. Plant Physiol. 76, 607-611.

Mauch-Mani, B., and Slusarenko, A. J. (1994). Systemic acquired resistance in Arabidopsis thaliana induced by a predisposing infection with a pathogenic isolate of Fusarium oxysporum. Mol. Plant-Microbe Interact 7, 378-383.

Meins, F., Neuhaus, J. M., Sperisen, C., and Ryals, J. (1991). The primary structure of plant pathogenesis-related glucanohydrolases and their genes. In Genes involved in plant defence, T. Boller, F. Meins, eds. (Springer Verlag, Berlin), pp. 245-282.

Nairn, C. J., Winsett, L., and Ferl, R. J. (1988). Nucleotide sequence of an actin gene from Arabidopsis thaliana. Gene 65, 247-257.

P\tilde{e}na-Cortes, H., Albrecht, T., Prat, S., Weiler, E. W., and Willmitzer, L. (1993). Aspirin prevents wound-induced gene expression in tomato leaves by blocking jasmonic acid biosynthesis. Planta 191, 123-128.

Penninckx et al. (1996) Plant Cell 8, 2309-2323.

Potter, S., Uknes, S., Lawton, K., Winter, A. M., Chandler, D., Dimaio, J., Novitzky, R., Ward, E., and Ryals, J. (1993). Regulation of a hevein-like protein in,Arabidopsis. Mol. Plant-Microbe Interact. 6, 680-681.

Sambrook, J., Fritsch, E. F., and Maniatis, T. (1989). Molecular cloning: A Laboratory Manual. Cold Spring Harbor Laboratory Press, Cold Spring, Harbor, N.Y.

Schaller, G. E., and Bleecker, A. B. (1995). Ethylene-binding sites generated in yeast expressing the Arabidopsis ETRl gene. Science 270, 1809-1811.

Schweizer et al (1997) Plant Physiol. 114, 79-88.

Terras, F. R. G., Schoofs, H. M. E., De Bohle, M. F. C., Van Leuven, F., Rees, S. B., Vanderleyden, J., Cammue, B. P. A., and Broekaert, W. F. (1992). Analysis of two novel classes of plant antifungal proteins from radish (Raphanus sativzus L.) seeds. J. Blol. Chem. 267, 15301-15309.

Terras, F. R. G., Torrekens, S., Van Leuven, F., Osborn, R. W. Vanderleyden, J. Cammue, B. P. A., and Broekaert, W. F. (1993). A new family of basic cysteine-rich plant antifungal proteins from Brassicaceae species. FEBS Lett. 316, 233-240.

Terras, F.R.G., Eggermont, K., Kovaleva, V., Raikhel, N. V., Osborn, R. W., Kester, A., Rees, S. B., Torrekens, S., Van Leuven, F., Vanderleyden, J., Cammue, B. P. A., and Broekaert, W. F. (1995). Small cysteine-rich antifungal proteins from radish: Their role in host defence. Plant Cell 7, 573-588.

Uknes, S., Mauch-Mani, B., Moyer, M., Potter, S., Williams, S., Dincher, S., Chandler, D., Slusarenko, A., Ward, E., and Ryals, J. (1992). Acquired resistance in Arabidopsis. Plant Cell 4, 645-656.

Uknes, S., Winter, A., Delaney, T., Vernooij, B., Morse, A., Friedrich, L., Nye, G., Potter, S., Ward, E. and Ryals, J. (1993). Biological induction of systemic acquired resistance in Arabidopsis. Mol. Plant-Microbe Interact. 6, 692-698.

Valvekens, D., van Montagu, M. and van Lijsebettens, M., (1988), Agrobacterium tumefaciens mediated transformation of Arabidopsis thaliana root explants by using kanamvcin selection. Pros Natl.-Acad. Sci. USA. 85, 5536-5540.

Warner, S. A. J., Scott, R., and Draper, J. (1993). Isolation of an asparagus intracellular PR gene (AoPR1) wound-responsive promoter by the inverse polymerase chain reaction and its characterization in transgenic tobacco. Plant J. 3, 191-201.

Ward, E. R., Uknes, S. J., Williams, S. C., Dincher, S. S., Wiederhold, D. L., Alexander, D. C., Ahl-Goy, P., Métraux, J.-P., and Ryals, J. (1991). Coordinate gene activity in response to agents that induce systemic acquired resistance. Plant Cell 3, 1085-1094.

White et al (1979) Virology, 99, 410-412.

1. A method of protecting a plant against a pathogen, the method comprising inducing expression of a plant defensin gene by stimulating the jasmonate and/ or ethylene pathways.

2. A method according to claim 1 wherein expression of a plant defensin gene or a coordinately expressed gene is induced by stimulating the jasmonate and ethylene pathways.

3. A method according to claim 1 or claim 2 wherein the pathogen is necrotrophic pathogen.

4. A method according to any one of claims 1 to 3 wherein the pathogen is a microbial pathogen.

5. A method according to any one of the preceding claims wherein the pathogen is a fungus.

6. A method according to any one of the preceding claims wherein the pathways are stimulated by the application of one or more of ethylene, jasmonicacid or alas jasmonate-like compound, an agent which mimics the action of ethylene, an agent which mimics the action ofjasmonic acid, a non-herbicidal amount of an agent which mimics the action of ethylene, a non-herbicidal amount of an agent which mimics the action ofjasmonate and an agent which causes oxidative stress.

7. A method according to any one of the preceding claims wherein stimulation of the jasmonate and/ or ethylene pathways involves the signal traasduction components EIN 2 and COI 1 from Arabidopsis.

8. A method of inducing expression of a plant defensin gene by applying to the plant one or more of ethylene, jasmonate. an agent which mimics the action of ethylene or jasmonate and an agent which causes oxidative stress.

9. A method according to claim 7 or claim 8 wherein the agent capable of causing oxidative stress is a diphenyl herbicide such as paraquat or diquat which result in the formation of superoxide anions or rose bengal or eosine which leads to the production of singlet oxygen species.

10. A composition which is capable of inducing expression of a plant defensin gene comprising one or more ofjasmonic acid, a jasmonate, ethylene. an agent which mimics the action of ethylene or jasmonate and an agent which causes oxidative stress.

11. A composition which is capable of inducing expression of a plant defensin gene comprising one or more of an ethylene-generating compound, a lipid derived signal molecule, salicylic acid, functional analogues of salicylic acid and reactive oxygen generating compounds.

12. A composition according to claim 11 wherein the ethylene-generating compound is selected from ethylene. ethephon and aminocyclopropanecarboxylic acid.

13. A composition according to claim 11 or claim 12 wherein the lipid-derived signal molecule is selected from arachidonic acid and derivatives thereof, linolenic acid and derivatives thereof and jasmonate and derivatives thereof.

14. A composition according to any one of claims 11 to 13 wherein the reactive oxygen generating compound is selected from paraquat, diquat, rose bengal and eosine.

15. A method for screening compounds for resistance-inducing activity, the method comprising applying to a plant or part of a plant a compound suspected of giving such resistance and detecting the expression of a plant defensin gene or a coordinately expressed gene.

16. A method according to claim 15 wherein the pathogen is a necrotrophic pathogen.

17. A method according to claim 15 or claim 16 wherein the pathogen is a microbial pathogen.

18. A method according to any one of claims 15 to 17 wherein the pathogen is a fungus.

19. A method according to any one of claims 15 to 18 wherein the plant is radish, tobacco or Arabidopsis.

20. A method according to claim 19 wherein the plant is Arabidopsis and the defensin is the product of the plant defensin gene PDF 1.2 (FIG. 14), a sequence that has substantial homology with the sequence of PDF1.2 or a variant thereof.

21. A method according to any one of claims 15 to 20 wherein the compound is applied to a leaf.

22. A method according to any one of claims 15 to 21 wherein the detection is carried out by using antibodies against the gene products of PDF-1.1 or PDF 1.2.

23. A promoter which is capable of inducing the expression of a plant defensin gene comprising a region which is induced by jasmonic acid or an agent which mimics the action thereof and/ or ethylene or an agent which mimics the action thereof.

24. A promoter region comprised within the nucleic acid sequence shown in FIG. 14, or a sequence that has substantial homology with that shown in FIG. 14. or a variant thereof.

25. A method, a composition and a promoter substantially as hereinbefore described with reference to any one of the Figures.
